(19) United States
(12) Patent Application Publication
(10) Pub. No.: US 2025/0120921 A1
(43) Pub. Date: Apr. 17, 2025

(54) A MECHANO-RESPONSIVE NANOFIBROUS PATCH FOR THE DELIVERY OF BIOLOGICS IN LOAD-BEARING TISSUES

(71) Applicants: THE TRUSTEES OF THE UNIVERSITY OF PENNSYLVANIA, Philadelphia, PA (US); THE UNITED STATES GOVERNMENT AS REPRESENTED BY THE DEPARTMENT OF VETERANS AFFAIRS

(72) Inventors: Robert Leon MAUCK, Philadelphia, PA (US); Harvey E. SMITH, Philadelphia, PA (US); Ana Paula PEREDO NERY, Miami, FL (US); Sarah Elizabeth GULLBRAND, Flourtown, PA (US); Daeyeon LEE, Wynnewood, PA (US); George R. DODGE, Philadelphia, PA (US)

(21) Appl. No.: 18/834,436

(22) PCT Filed: Feb. 3, 2023
(86) PCT No.: PCT/US2023/061929

§ 371 Date: Jul. 30, 2024

Related U.S. Application Data
(60) Provisional application No. 63/306,647, filed on Feb. 4, 2022.

(51) Int. Cl. A61K 9/70; A61K 38/20; C07K 16/22

(52) U.S. Cl. CPC A61K 9/7084; A61K 38/2006; C07K 16/22

(57) ABSTRACT

Provided are localized therapeutic delivery articles, comprising: a first fibrous layer comprising a first plurality of nanofibers; and a second fibrous layer comprising a second plurality of nanofibers, the first fibrous layer and the second fibrous layer being sealed to one another so as to define at least one sealed compartment therebetween, the article optionally comprising a first population of mechanically-responsive delivery particles configured to rupture upon exposure to a first rupture force, the first population of mechanically responsive delivery particles being disposed within at least one sealed compartment. The nanofibers can be aligned, which alignment allows the layers to emulate the structure of various body tissues, thereby affording control over cell growth on the articles. Also provided are methods of using and fabricating the disclosed articles.

### CROSS-REFERENCE TO RELATED APPLICATIONS

The present application claims priority to and the benefit of U.S. patent application No. 63/306,647, “Mechano-Responsive Nanofibrous Patch For The Delivery Of Biologics In Load-Bearing Tissues” (filed Feb. 4, 2022). All foregoing applications are incorporated herein by reference in their entireties for any and all purposes.

### GOVERNMENT RIGHTS

This invention was made with government support under AR071340 awarded by the National Institutes of Health and IK6 RX003416, IK2 RX003118, IK2 RX001476, 101 RX002274 and I21 RX003447 awarded by the Department of Veterans Affairs. The government has certain rights in the invention.

### TECHNICAL FIELD

The present disclosure relates to the field of localized delivery of therapeutics and to the field of mechanically-responsive particles.

### BACKGROUND

Drug delivery systems exist to support a variety of physiologic functions and to modify a biologic process via the provision of select molecules to targeted sites. Common challenges for effective drug delivery are physical barriers (e.g., blood-brain barrier, cell membrane barrier), location of the target tissue, drug transport phenomena such as clearance dynamics, and short drug bioactivity, among others. Sustained release mechanisms provide for the extended presence of a drug; however, synchronizing the timing of drug release with the specific needs of the tissue remains a challenge. Recently, more complex systems that leverage physiological stimuli, such as temperature and enzymatic activity, have emerged to control drug provision. These methods hold great promise for enhancing the therapeutic effect of the delivered biomolecules by tuning their release to the physiologic demands of the delivery environment. However, these methods do not take into consideration the specialized mechanically loaded environments of tissues such as the heart, skin, and musculoskeletal tissues.

The delivery of biologics for musculoskeletal tissue regeneration poses several challenges including penetration of the characteristic dense extracellular matrix and the complex mechanical loading environment. To prevent and treat musculoskeletal conditions, local drug delivery systems such as intramuscular, subcutaneous, or intraarticular drug injections exist, yet these methods remain inadequate due to short drug half-life, rapid clearance out of joints, and difficulty in penetrating into the dense tissue matrix. Moreover, these injections result in a rapid increase in local biomolecule concentration (to supra-physiologic levels), followed by fast clearance, providing short-lived benefits and thus requiring repeated drug administration. Additionally, as musculoskeletal tissues heal and mature, the needs of the tissue change, making sequential and timed delivery of multiple therapeutic agents imperative to optimize healing outcomes. Accordingly, there is a long-felt need in the field for an improved technology and related methods for local delivery of therapeutic agents.

### SUMMARY

Provided here is a delivery scaffold for drug delivery platforms (including mechanically-activated microcapsules-MAMCs, and other mechanically responsive drug delivery technologies), that enables force transfer between the tissue and scaffold to elicit the delivery of biomolecules from mechano-responsive drug encapsulating depots. This scaffold provides topographical cues that guide cellular infiltration and matrix deposition, enhancing its integration with the native tissue and mechanical support. Furthermore, it can be formed of a material that is biodegradable (over ˜1-2 years), requiring a single procedure for delivery without the need for extraction from the body. The scaffold material can be biocompatible and does not elicit a chronic inflammatory response, providing a safe administration of drug delivery technology.

The delivery of drug delivery platforms (e.g., MAMCs) to the target tissue remains a challenge for tissues that are not surrounded by naturally existing barriers (e.g. joint capsule) that can constrain the microcapsules for localized delivery. Therefore, we developed a nanofibrous scaffold for therapeutic delivery that encapsulates the drug delivery platform and enables the transfer of tissue strains to trigger mechano-activation in response to tissue mechanical loading.

In the present disclosure, MAMCs are referred to as an example drug delivery platform. MAMCs are used as illustrative only, however, and the present disclosure should not be understood as being limited to MAMCs. Example MAMCs are described in, e.g., United States patent application publication US 2018/0169024, the entirety of which is incorporated herein by reference for any and all purposes.

In meeting the described long-felt needs in the art, the present disclosure provides a localized therapeutic delivery article, comprising: a first fibrous layer comprising a first plurality of nanofibers; and a second fibrous layer comprising a second plurality of nanofibers, the first fibrous layer and the second fibrous layer being sealed to one another so as to define at least one sealed compartment therebetween, the article optionally comprising a first population of mechanically-responsive delivery particles configured to rupture upon exposure to a first rupture force, the first population of mechanically responsive delivery particles being disposed within at least one sealed compartment.

Also provided are methods, comprising introducing an article according to the present disclosure (e.g., according to any one of Aspects 1-16) to a subject, the introduction optionally being to an annulus fibrosus of the subject.

Further disclosed are methods, comprising causing the introduction of an article according to the present disclosure (e.g., according to any one of Aspects 1-16) to a subject, the introduction optionally being to the annulus fibrosus of the subject.

Additionally provided are methods, comprising causing application of a strain to an article according to the present disclosure (e.g., according to any one of Aspects 1-16).

Also disclosed are methods, comprising application of a strain to an article according to the present disclosure (e.g., according to any one of Aspects 9-16) so as to effect rupture of at least some of the first population of delivery particles.

Further provided are methods, comprising fabricating an article according to the present disclosure, e.g., according to any one of Aspects 1-16.

Also disclosed are methods, comprising: with a first fibrous layer comprising a first plurality of nanofibers and a second fibrous layer comprising a second plurality of nanofibers, sealing the first fibrous layer and the second fibrous layer to one another so as to define at least one sealed compartment therebetween.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the drawings, which are not necessarily drawn to scale, like numerals can describe similar components in different views. Like numerals having different letter suffixes can represent different instances of similar components. The drawings illustrate generally, by way of example, but not by way of limitation, various aspects discussed in the present document. In the drawings:

FIGS. 1A-1G. (A) Mechano-responsive repair patch assembly, with variations in 8. (B) Quantification and (C) visualization of MAMCs popped with melt-stamping (scale: 1 mm, 100 μm inset). (D) Optical strain tracking example (scale: 1 mm) and (E) quantification of strain transfer in direction of applied deformation. (F) Quantification and (G) visualization of MAMCs popped with loading (scale: 100 μm).

FIGS. 2A-2E. (A) Anakinra and Tanezumab MAMCs (scale: 50 μm). (B) Gene expression normalized to AF cells grown in basal media. (C) PC-12 cell nerve density, (D) branching, and (E) length with soluble or MAMC Tanezumab.

FIGS. 3A-3C. (A) Annular injury via partial thickness AF laceration (l: 5 mm, w: 2.5 mm, d: 4 mm) and full thickness needle puncture (14G needle; ø: 2.1 mm). (B) Surgical image of injury and patch repair (scale bar: 1 mm). (C) linear modulus.

FIGS. 4A-4D: (a) Anakinra delivery blocks IL-18 signaling. (b) Anakinra-loaded MAMCs (scale: 50 μm). (c) Alamar Blue viability assay for cells treated with Anakinra. (d) MMP3 and LCN2 expression after treatment with soluble Anakinra or Anakinra extracted from crushed MAMCs in the presence of IL-1β.

FIGS. 5A-5C. (a) Schematic of melt-stamped scaffolds. (b) MAMC mechano-activation with tensile loading (# vs. 0 cycles, * vs 3 mm) and (c) scaffold tensile modulus.

FIGS. 6A-6D: (a) Linear modulus normalized to healthy control (dashed line). (b) Mallory Heindenhain-stained section for empty scaffold repair (dashed line) (scale: 1 mm). Fibrous tissue infiltration area in the (c) anterior and (d) posterior AF.

FIG. 7 describes that surgical intervention of symptomatic disc can leave the disc unrepaired.

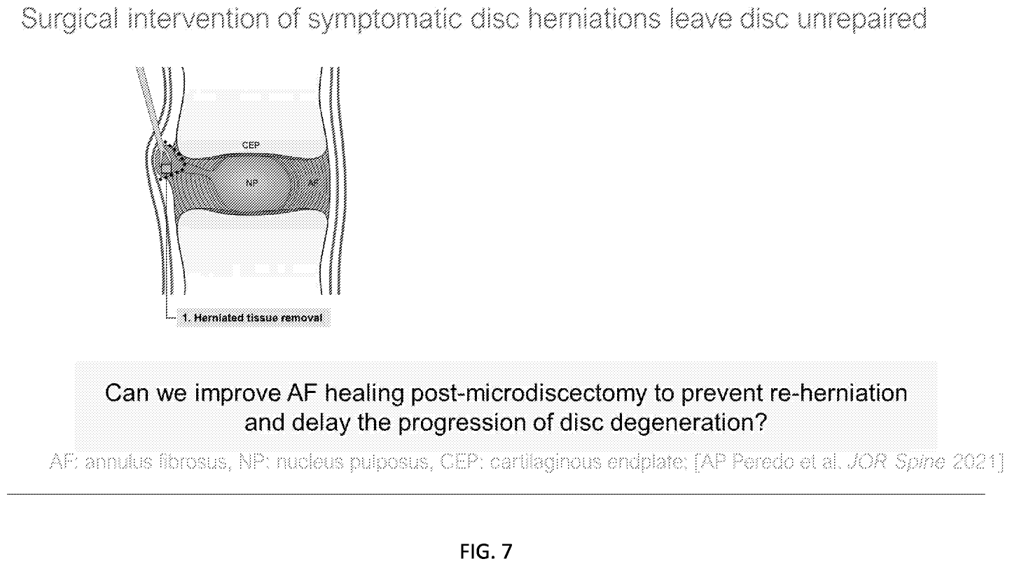

FIG. 8 presents certain impediments to proper healing and restoration of the AF.

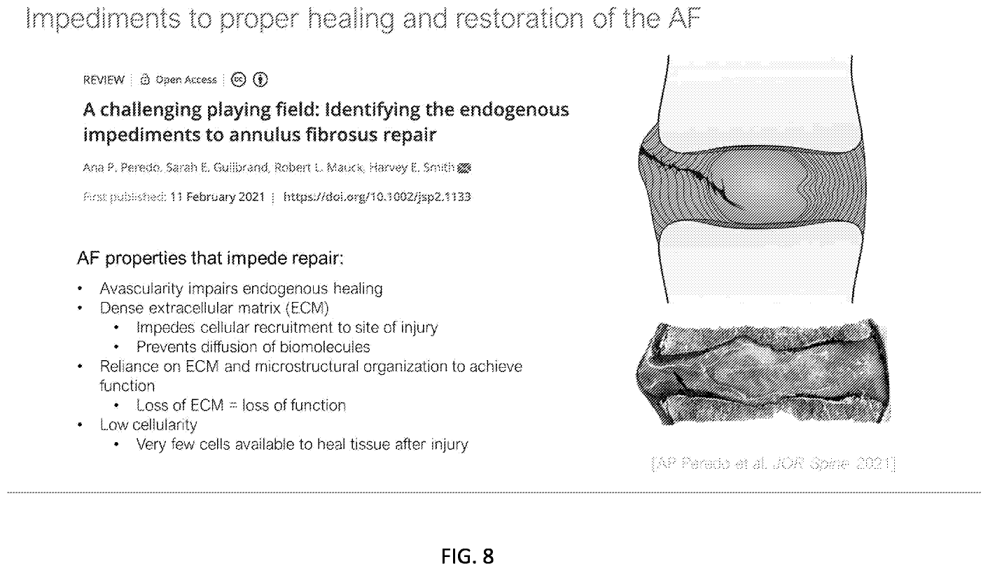

FIG. 9 illustrates scaffold fabrication via melt stamping, in accordance with the present disclosure.

FIG. 10 illustrates the melt stamping can increase scaffold strength and toughness.

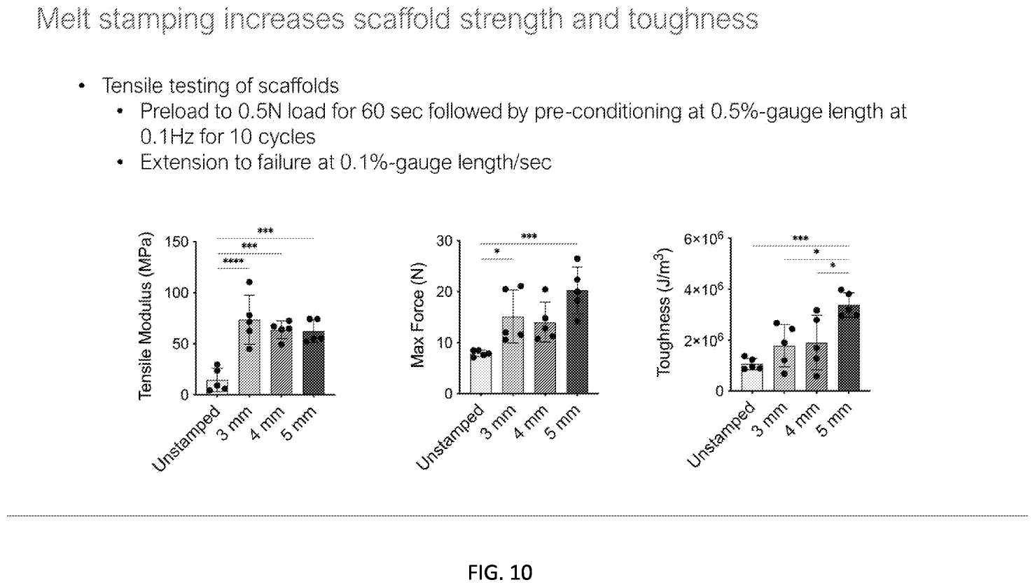

FIG. 11 illustrates MAMC mechanoactivation under dynamic tensile loading.

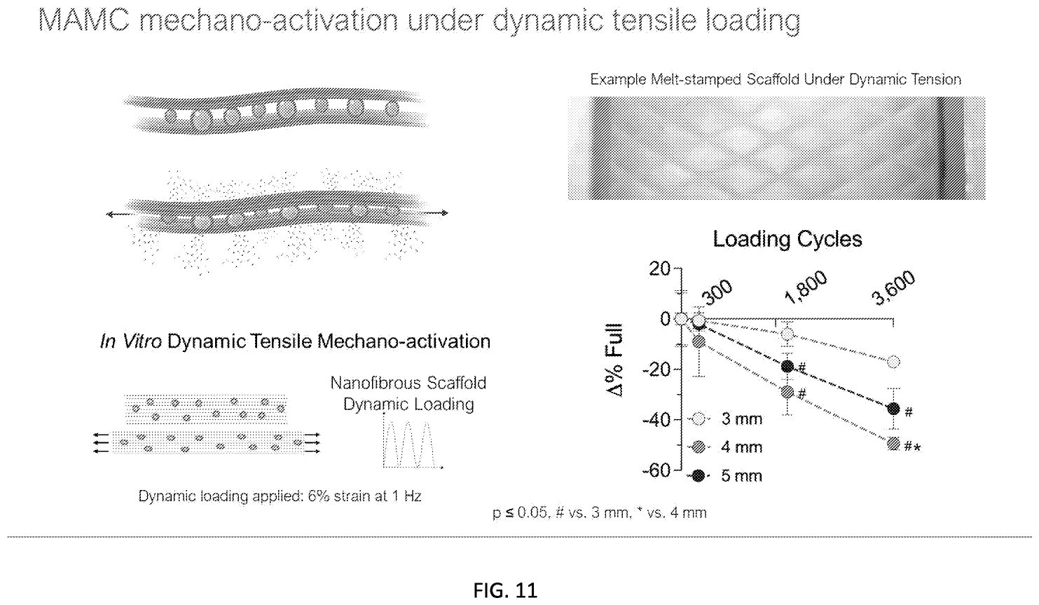

FIG. 12 illustrates cellular sensing on melt-stamped scaffolds.

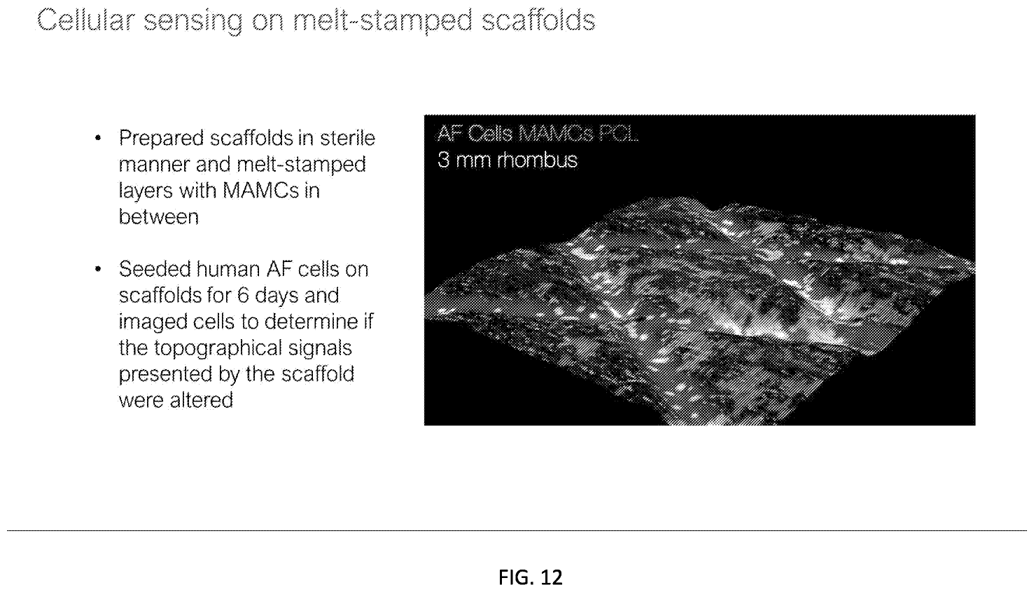

FIG. 13 illustrates that stamping of scaffolds increased cellular aspect ratio but preserved their alignment along fibers.

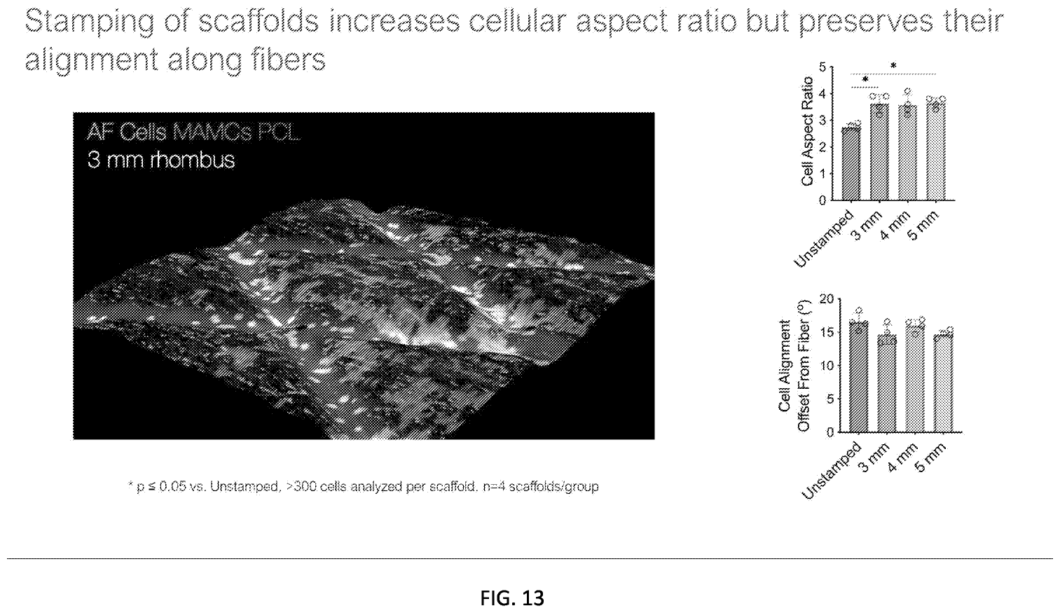

FIG. 14 illustrates a cell instructive bioactive scaffold for AF repair.

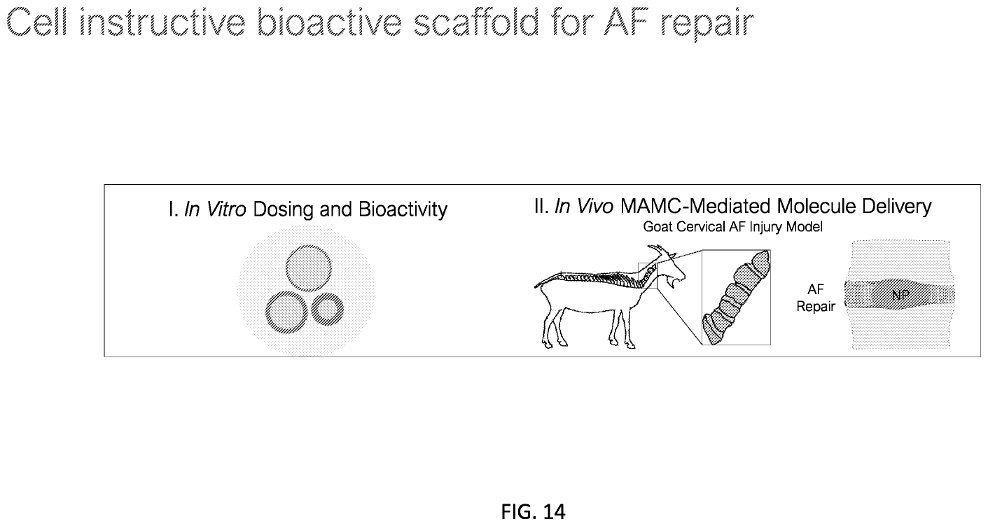

FIG. 15 illustrates a MAMC-mediated molecule delivery.

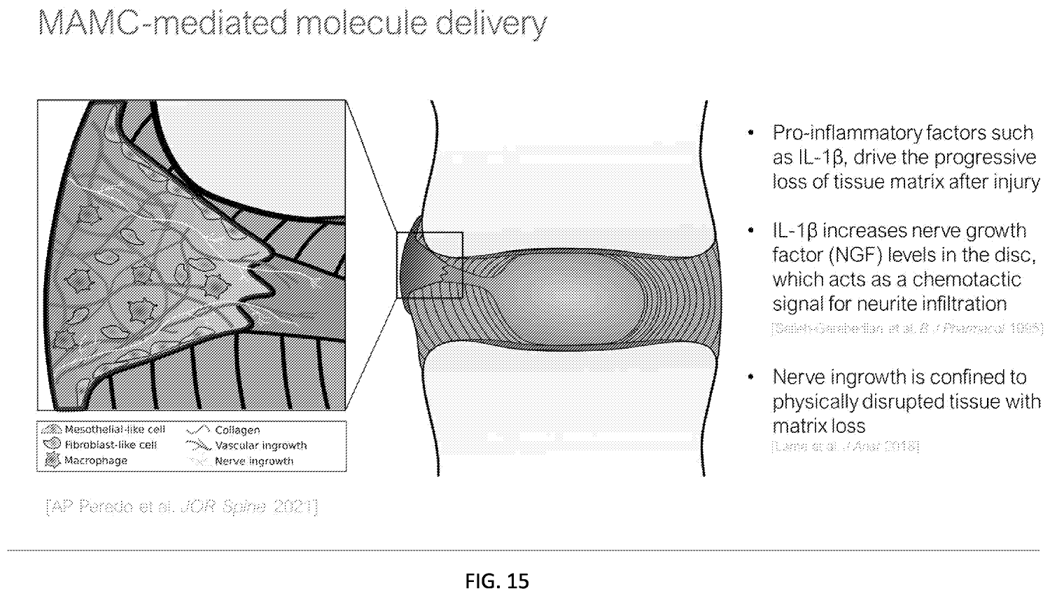

FIG. 16 illustrates in vitro MAMC-mediated molecule delivery.

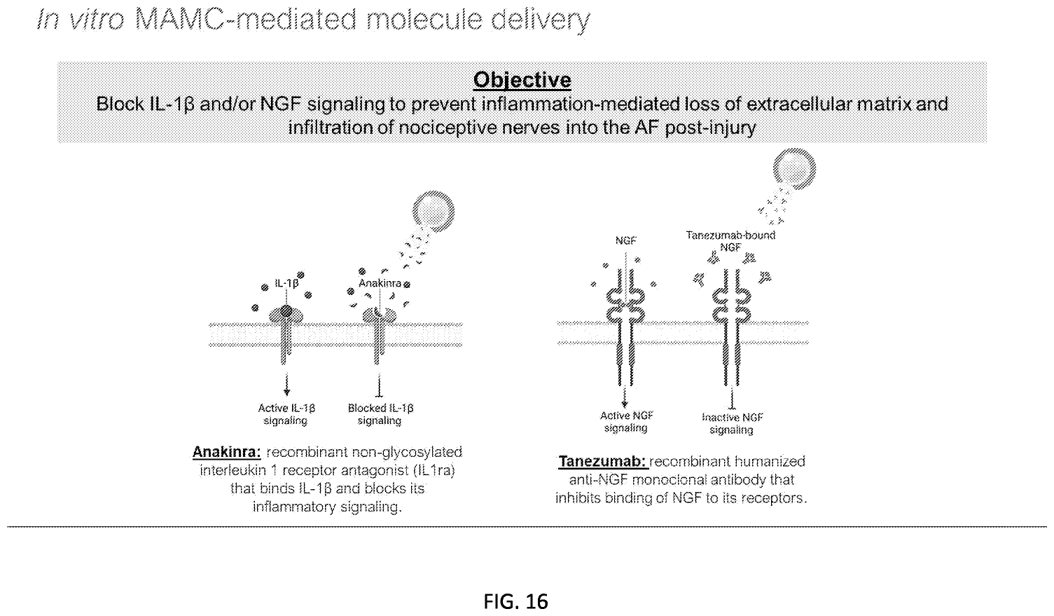

FIG. 17 illustrates in vitro MAMC-mediated Anakinra delivery to AF cells.

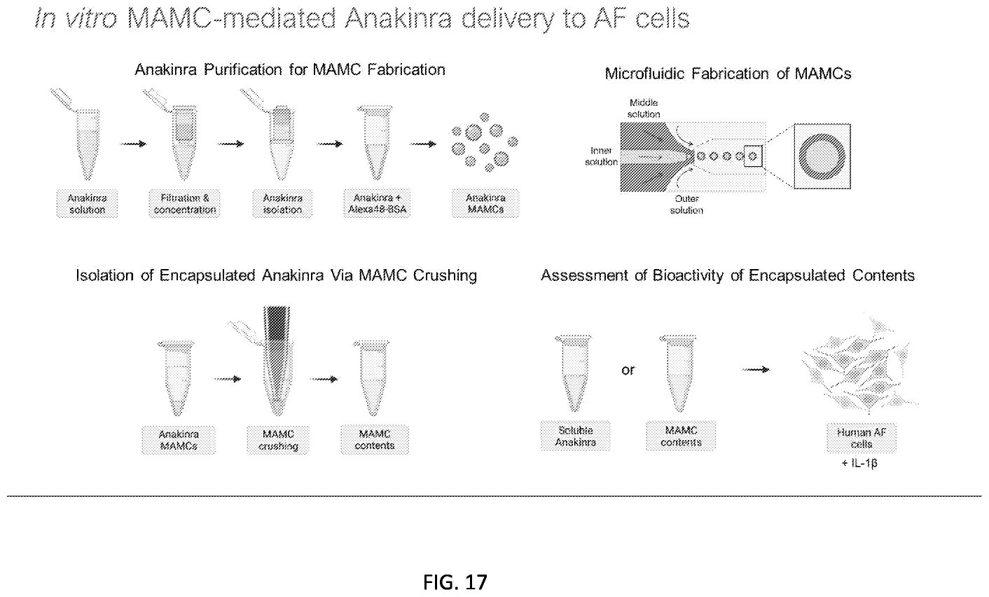

FIG. 18 illustrates that MAMC-mediated Anakinra delivery demonstrated bioactivity and dose-dependent effects.

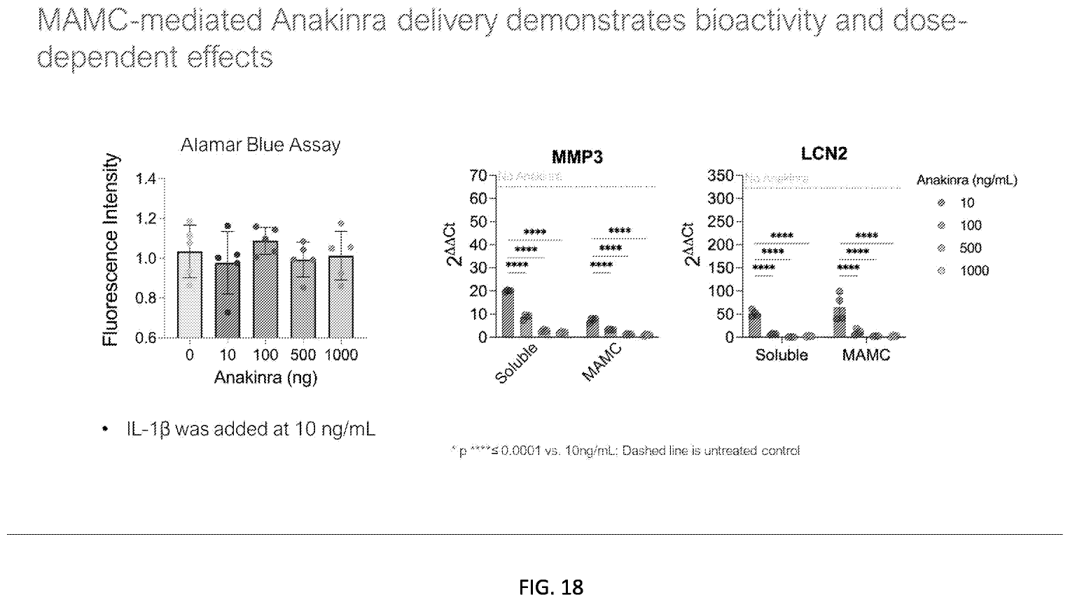

FIG. 19 illustrates that MAMC-mediated Tanezumab delivery prevents NGF-mediated neurite outgrowth.

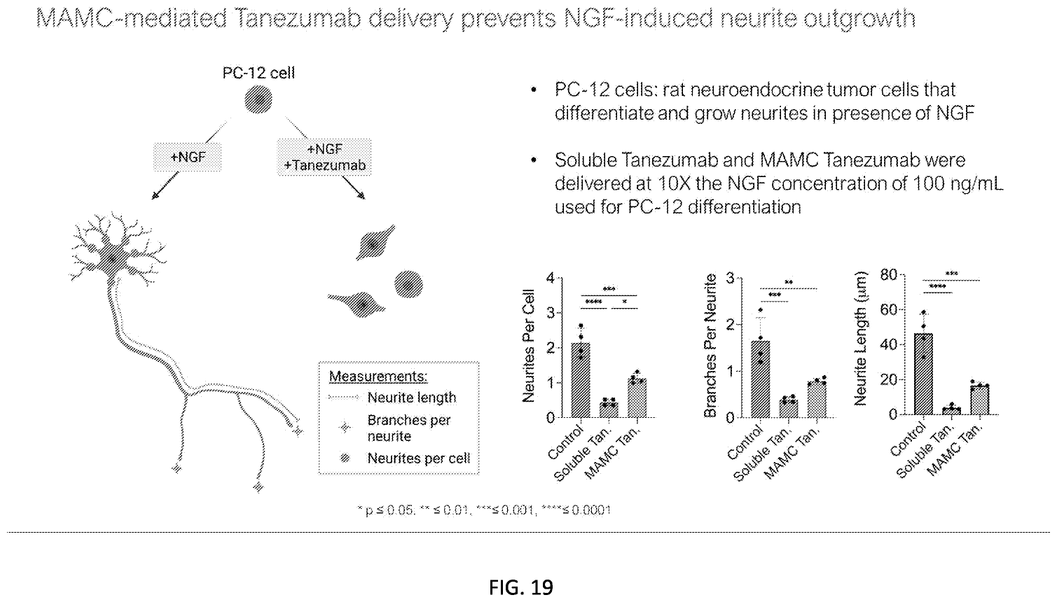

FIG. 20 illustrates the goat cervical spine for in vivo evaluation of a patch according to the present disclosure.

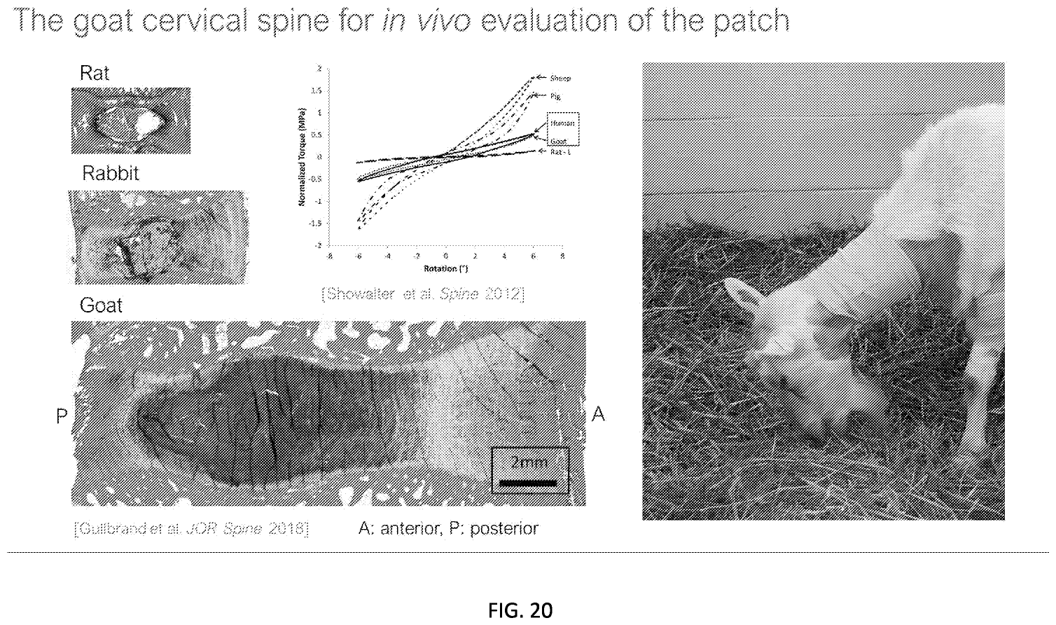

FIG. 21 illustrates in vivo MAMC-mediated molecule delivery.

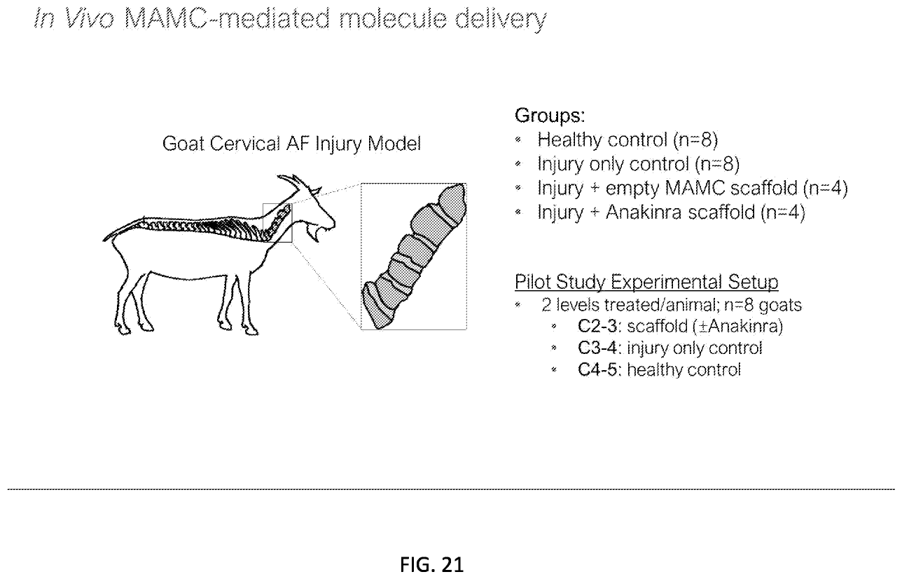

FIG. 22 illustrates an in vivo injury model of AF herniation in the goat anterior cervical disc.

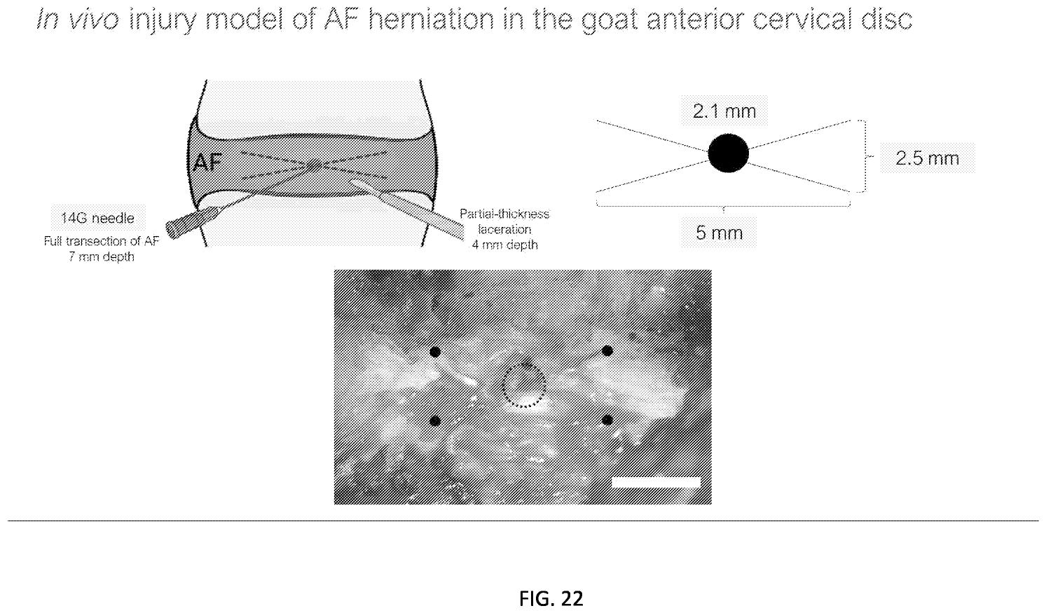

FIG. 23 illustrates in vivo delivery of a mechano-active patch according to the present disclosure for injury repair.

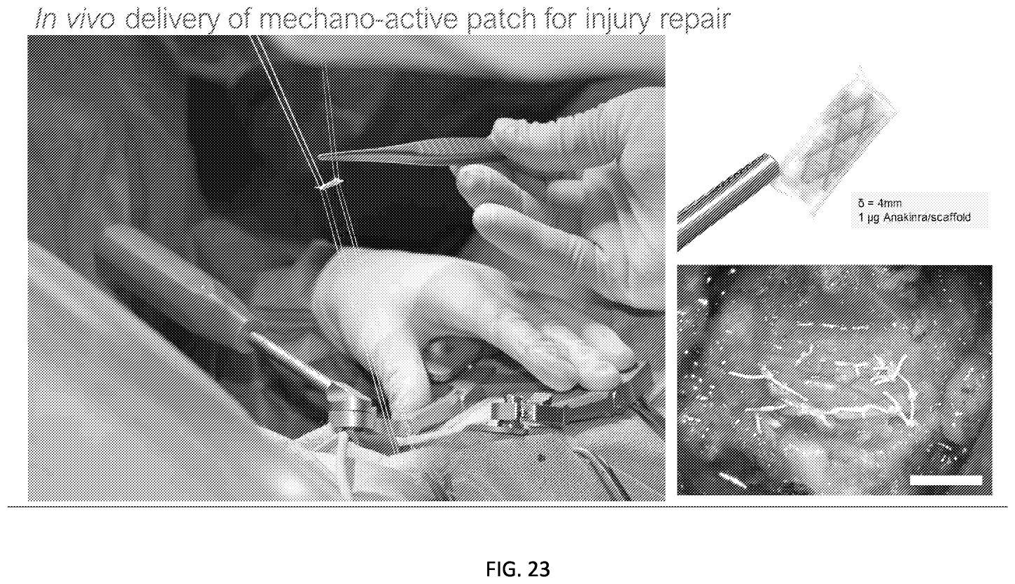

FIG. 24 illustrates that MRI T2 imaging reveals disc remodeling with injury. In particular, the MRI data at FIG. 24 illustrate the effectiveness of the disclosed technology, as an injured subject receiving a patch that includes Anakinra (A-Scaffold) exhibited T2 levels at (uninjured) control levels.

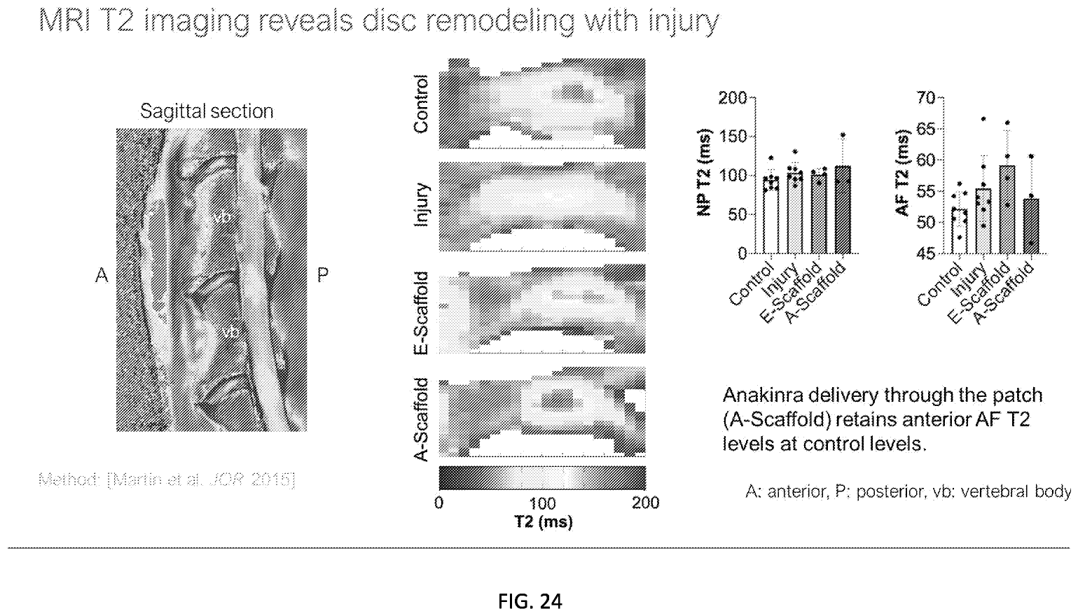

FIG. 25 illustrates that an empty patch according to the present disclosure slightly attenuates injury-induced disc stiffening.

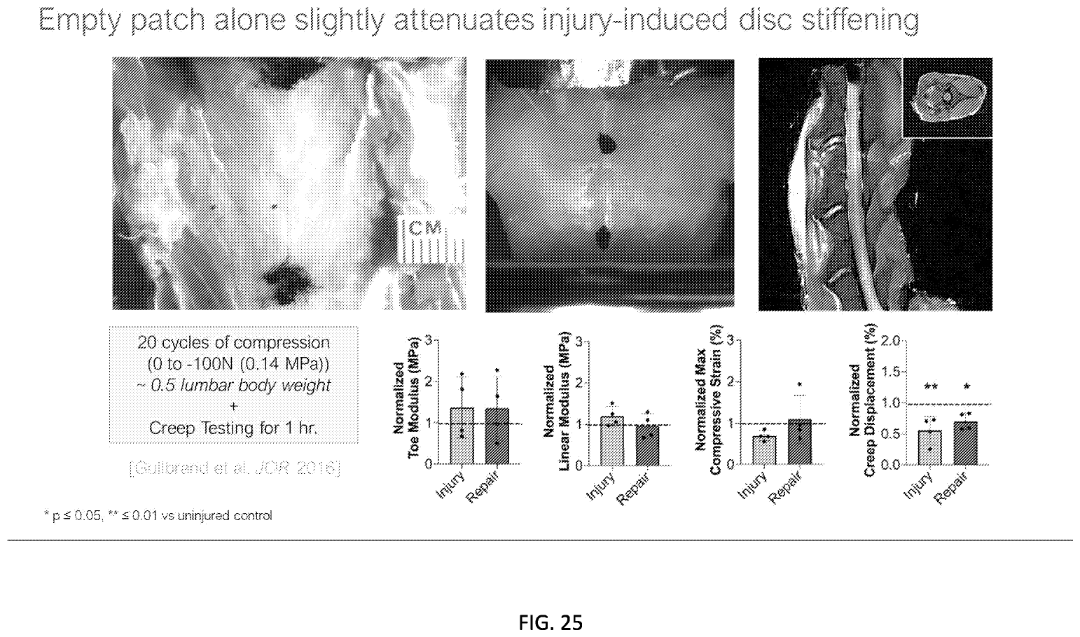

FIG. 26 provides a histological analysis of biochemical content and scar formation for an untreated control, an injury-only control, and an empty patch control.

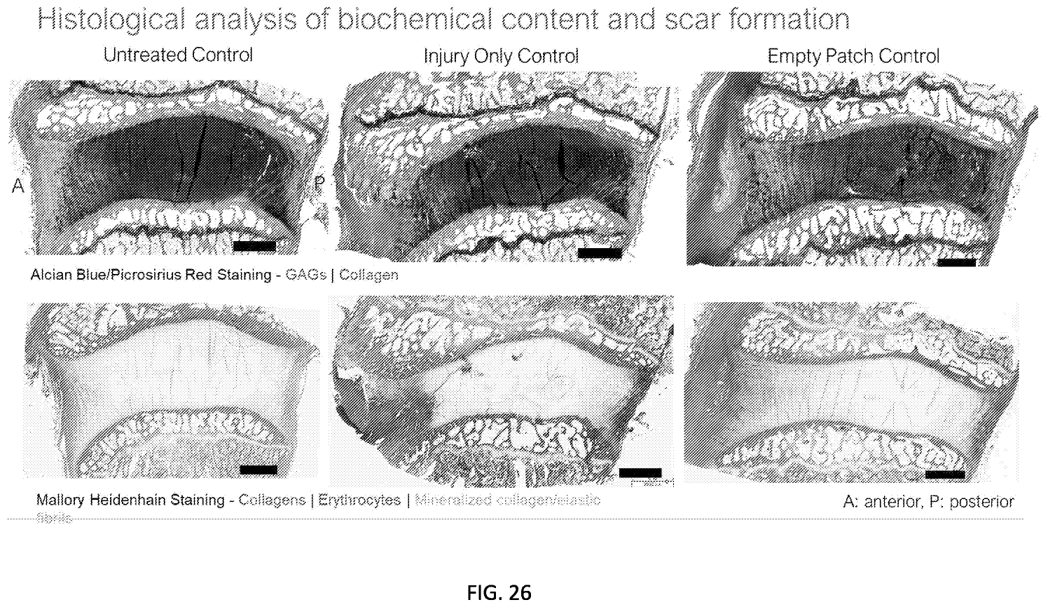

FIG. 27 illustrates collagen scar infiltration measurements form histological slides.

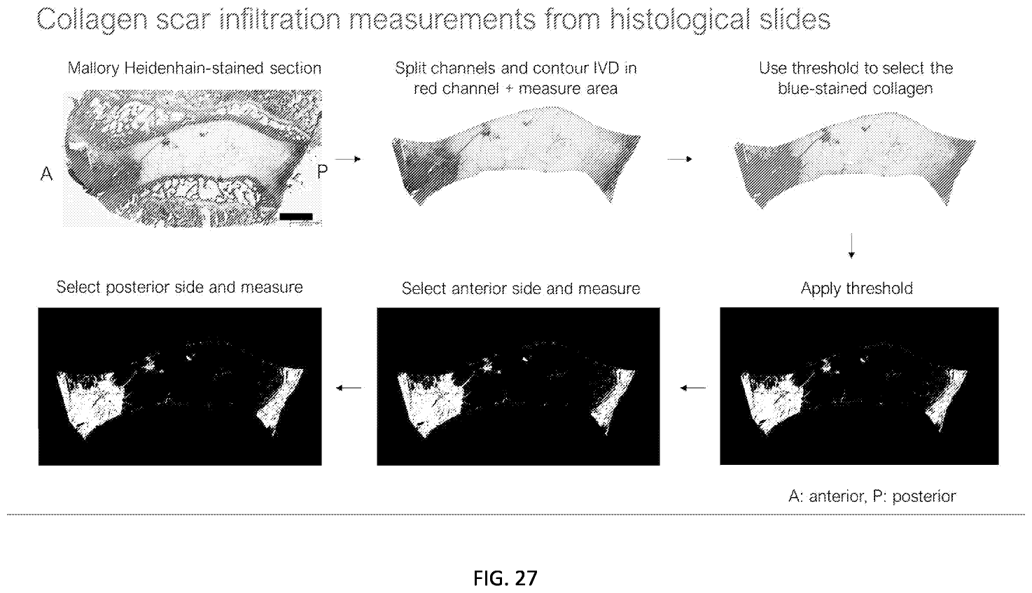

FIG. 28 illustrates that patch delivery prevents infiltration on both anterior/posterior AF.

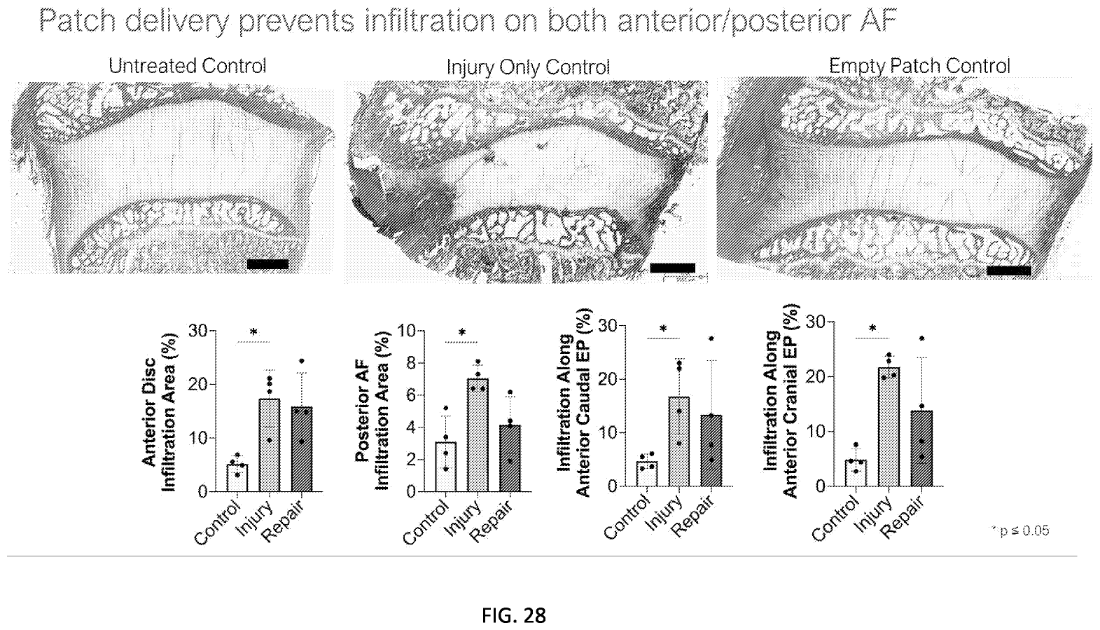

FIGS. 29A-29G depict TARP fabrication with different stamp geometries regulates MAMC patency and scaffold biomechanical properties. (FIG. 29A) Schematic of MAMC fabrication using a glass capillary microfluidic device. (FIG. 29B) Schematic of TARP fabrication via melt-stamping using stamps of different rhombus patterns defined by the longest rhombus diagonal length (d). (FIG. 29C) TARP after melt-stamping, encapsulated MAMCs seen in pink (magnified in the inset; red: MAMC shell, green: encapsulated cargo) (scale: 5 mm). (FIG. 29D) Interior of TARP imaged after melt-stamping, with MAMCs crushed/melted along the stamped region (white dashed line), verified by a loss of encapsulated cargo (green) and deformation of the MAMC shell (red) (scale: 1 mm, inset scale: 100 mm). (FIG. 29E) MAMC patency, measured as % Full (mean±s.d.), after stamping with different stamp d (n=4 scaffolds/type). Starting % Full levels before melt stamping are indicated by the dashed line. (FIG. 29F) Stiffness and (FIG. 29G) maximum force for unstamped scaffolds or TARPs of different stamp d (mean±s.d.) (n=5/type). Statistical significance denoted by * p-value<0.05, **<0.01, ***<0.001.

FIGS. 30A-30E depict local strain transfer dictated by stamping pattern impacts MAMC mechano-activation with dynamic physiologic tensile loading. (FIG. 30A) Schematic of optical tracking of marked rhombus vertices for local strain measurements on unstamped scaffolds and TARPs during the application of tensile ramp to failure. (FIG. 30B) Local strain in unstamped scaffolds and TARPs along the longitudinal fiber/loading direction (e_{y}) or transverse direction (eₓ) inside marked rhombi plotted versus applied Instron strain (mean±s.d.) (n=5/type). Physiologic outer annulus fibrosus strain (6%) highlighted by black dashed line. (FIG. 30C) Schematic of mechano-activation of MAMCs within the TARPs upon the application of dynamic tensile loading. (FIG. 30D) Representative images of MAMCs in TARP (d=4 mm) before the application of dynamic loading (0 cycles) or after 3,600 cycles of uniaxial tension (scale: 100 mm). Mechano-activation of MAMCs is visualized through the loss of cargo (green) from MAMCs (shell outlined in red) with TARP loading. (FIG. 30E) MAMC mechano-activation (D % Full, mean±s.d.) for dynamic physiologic tensile loading (6% strain, 1 Hz) of TARPs (n=4/type) as a function of applied loading cycles. Statistical significance denoted by #p-value<0.05 vs. 3 mm, * p-value<0.05 vs 4 mm.

FIGS. 31A-31E depict in vivo repair via TARP implantation in a goat cervical disc annular injury model. (FIG. 31A) Study design for the in vivo study using a goat cervical disc annular injury model. (FIG. 31B) Depiction of the annular injuries induced on the anterior aspect of the intervertebral disc. (FIG. 31C) Intra-operative photograph of the anterior cervical disc after annular rupture, with vertices of the X-laceration demarcated by black dots and the needle puncture area demarcated with a dashed black circle (scale: 5 mm). (FIG. 31D) Intra-operative photograph of the cervical disc after repair with a TARP, sutured across all edges to the native tissue (scale: 5 mm). (FIG. 31E) Photograph showing pre-threading of sutures through the corners of the cruciate annulotomy and the TARP being advanced along the sutures in order to secure each corner at the X-laceration and ensure central placement across the injury.

FIGS. 32A-32F depict TARPs show robust integration with native tissue. (FIG. 32A) Representative H&E-stained sagittal section and zoom in of anterior AF for cervical discs in the uninjured control, (FIG. 32B) injury, (FIG. 32C) E-scaffold-treated, and (FIG. 32D) A-scaffold-treated groups (scale: 2 mm, inset scale: 1 mm). (FIG. 32E) Representative H&E-stained section for E-scaffold and (F) A-scaffold-treated discs, showing cellular infiltration of the empty and Anakinra-loaded TARPs with robust extracellular matrix deposition throughout the scaffold and integration with host tissue (scale: 100 μm). MAMC outlines (black asterisk) are apparent inside Anakinra-loaded TARP.

FIGS. 33A-33F depict effects of TARP repair and Anakinra delivery on disc T2 signal and biochemical composition. (FIG. 33A) Average sagittal T2 maps, with dimensions normalized to account for disc curvature and anatomical differences, for levels with injury (n=7), repair with empty TARP (E-Scaffold, n=4), and repair with Anakinra-loaded TARP (A-Scaffold, n=3). (FIG. 33B) Average T2 (mean±SEM) at central Y axis (y=0) of normalized T2 maps along the normalized X dimension. Anterior AF and NP regions measured through ROIs, quantified in C-D appear shaded in. (FIG. 33C) AF and (FIG. 33D) NP T2 measurements (mean±s.d.) for rectangular and circular regions of interest, respectively, for treated discs compared to uninjured controls (dashed line: control mean). (FIG. 33E) Alcian Blue/Picrosirius Red-stained sagittal sections for discs in each group with the highest NP Alcian Blue staining intensity (scale: 2 mm). (FIG. 33F) NP Alcian Blue intensity measurements (mean±s.d.) for sections from treated groups compared to uninjured control discs (dashed line). Statistical significance compared to control denoted by ** p-value<0.01.

FIGS. 34A-34G depict collagenous infiltration through the injury tract increases with Anakinra delivery. (FIG. 34A) Representative Mallory Heidenhain-stained and zoomed-in H&E-stained sagittal section of anterior AF for control, (FIG. 34B) injury, (FIG. 34C) E-Scaffold, and (FIG. 34D) A-Scaffold treated discs (scale: 2 mm; TARPs are outlined with black dashed line; inset scale: 50 μm). (FIG. 34E) Anterior AF infiltration area of collagenous scar/remodeling, stained in purple, measured as percentage of total disc area from Mallory Heindenhain-stained sections (mean±s.d.). Statistical significance denoted by ** p-value<0.01. (FIG. 34F) Linear modulus and (FIG. 34G) creep displacement for motion segments in each group compared to uninjured controls (dashed line: control mean). Statistical significance denoted by * p-value<0.05 vs uninjured control.

FIGS. 35A-35C illustrate that TARP implantation prevents disc-wide aberrant remodeling and tissue necrosis. (FIG. 35A) Representative H&E-stained sagittal histological sections, zoomed in on the posterior AF for control, injury, E-Scaffold, and A-Scaffold treated discs (scale: 50 μm). (FIG. 35B) Average number of nuclei per ROI in the posterior AF, measured from 3 ROIs along the outer AF of each section, using H&E-stained sagittal sections (mean±s.d.). (FIG. 35C) Posterior AF infiltration area of collagenous scar/remodeling, stained in purple, measured as a percentage of total disc area using Mallory Heindenhain-stained sagittal sections (mean±s.d.). Statistical significance denoted by * p-value<0.05, **<0.01.

FIGS. 36A-36E depict TARP fabrication and local strain measurements. (FIG. 36A) Schematic of fabrication of PCL-PEO nanofibrous scaffolds through simultaneous electrospinning and collection of fibers composed of each material on a rotating mandrel. (FIG. 36B) 3D-printed metal stamps displaying rhombus grids with varying rhombus geometries through the change in longest rhombus diagonal (δ), used for melt-stamping of TARPs. (FIG. 36C) Representative images of melt-stamped PCL-PEO layers with δ=3 mm (left) and δ=5 mm (right) (scale: 1 mm). (FIG. 36D) Local longitudinal strain (ε_{y}) and (E) transverse strain (εₓ) (mean±s.d.) in each rhombus at an applied Instron strain of 6% for unstamped scaffolds vs stamped groups (n=5/type).

FIGS. 37A-37F depict the effect of melt-stamping and rhombus geometry on fiber and cellular alignment. (FIG. 37A) 3D reconstruction of confocal z-stack for δ=3 mm TARP seeded with human AF cells (blue: nanofibers, green: f-actin, red: MAMC shells) (scale: 500 μm). (FIG. 37B) Fiber alignment offset from 90° and fiber angle dispersity (mean±s.d.) for unstamped and stamped scaffolds (n=4 scaffolds/type, with n=4 ROIs/scaffold). (FIG. 37C) Scaffold fiber orientation maps for unstamped and stamped TARPs with fibers colored based on orientation angle (scale: 50 μm). (FIG. 37D) Cell alignment offset from fiber direction and (FIG. 37E) cell aspect ratio (mean±s.d.) for cells seeded on scaffolds (n=4 scaffolds/type, with n=4 ROIs/scaffold). (FIG. 37F) Representative confocal image of cells seeded on a δ=4 mm TARP, with cell bodies (F-actin: red; nuclei: blue) oriented along the fiber direction (scale: 50 μm). Statistical significance denoted by #p-value<0.05 vs. unstamped for fiber alignment offset, * p-value<0.05 vs unstamped for fiber angle dispersity.

FIGS. 38A-38D illustrate MAMC-encapsulated Anakinra retains bioactivity and potently blocks IL-1b-mediated effects on human AF cells. (FIG. 38A) Schematic of the mechanism of MAMC-mediated Anakinra delivery to block IL-1b signaling. (FIG. 38B) Schematic of the comparison between direct delivery of soluble Anakinra and the contents of Anakinra-loaded MAMCs on human AF cells cultured with or without IL-1b. (FIG. 38C) Alamar Blue fluorescent intensity (mean±s.d.) of the media from AF cells cultured with different Anakinra concentrations for 6 days (n=5/treatment). (FIG. 38D) Relative gene expression (mean±s.d.) for MMP3 (left) and LCN2 (right) for AF cells cultured in the presence of IL-1b with or without different doses of Anakinra delivered via soluble direct delivery or extracted from Anakinra-loaded MAMCs (n=4/treatment). Orange dashed line represents gene expression for cells that did not receive Anakinra. Statistical significance denoted by * p-value ****<0.0001.

FIGS. 39A-39F illustrate TARP-mediated Anakinra delivery attenuates implantation-mediated osteolysis in adjacent vertebras. (FIG. 39A) Sagittal view of representative bone density maps for motion segments in each group, showing osteolysis in the vertebras adjacent to the E-Scaffold-treated disc (scale: 1 mm). (FIG. 39B) Osteolysis volume measured across groups on the cranial and caudal bony endplates of the motion segments (mean±s.d.). (FIG. 39C) Example contouring of cranial and caudal bony endplates along the sagittal plane that were morphed to created 3D contours for bone morphometric analysis in D-F (scale: 1 mm). (FIG. 39D) Bone volume to total volume ratio (BV/TV), (FIG. 39E) trabecular number (Tb. N.), and (FIG. 39F) trabecular spacing (Tb.Sp.) measured across groups on the cranial and caudal bony endplates of the motion segments (mean±s.d.). Cranial and caudal endplates from the same animal are shown as individual data points. Statistical significance denoted by * p-value<0.05, **<0.01.

FIGS. 40A-40G illustrate effects of annular injury on T2 and biomechanics. (FIG. 40A) Average T2 maps for each group showing level-matched controls (n=6) and injury (n=7). (FIG. 40B) AF and (FIG. 40C) NP T2 measurements for rectangular and circular regions of interest, respectively, for uninjured vs injured discs (mean±s.d.). (FIG. 40D) Toe modulus, (FIG. 40E) linear modulus, (FIG. 40F) maximum compressive strain, and (FIG. 40G) creep displacement (mean±s.d.) for motion segments in each group (n=7 and n=6 for uninjured control and injured discs, respectively). Statistical significance denoted by * p-value<0.05, **<0.01.

FIG. 41 illustrates biochemical compositional changes with annular injury and TARP repair. Alcian Blue-Picrosirius red-stained sagittal histological sections showing best, median, and worst specimens for each group, identified through NP Alcian Blue intensity measurements (scale: 2 mm).

FIG. 42 illustrates structural and compositional changes with annular injury and TARP repair. H&E-stained sagittal histological sections showing best, median, and worst specimens for each group, identified through NP Alcian Blue intensity measurements (scale: 2 mm.

FIG. 43 illustrates collagenous scar infiltration and remodeling differences with injury and treatment. Mallory Heindenhain-stained sagittal histological sections showing best, median, and worst specimens for each group, identified through NP Alcian Blue intensity measurements (scale: 2 mm).

FIGS. 44A-44C illustrate injury-induced stiffening of the disc is not reversed by TARP-implantation. (FIG. 44A) Depiction of mechanical testing setup for motion segment compression and creep testing. PBS bath not depicted. (FIG. 44B) Toe modulus and (FIG. 44C) maximum compressive strain (mean±s.d.) for motion segments in each group compared to uninjured controls (dashed line: control mean).

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints (e.g., “between 2 grams and 10 grams, and all the intermediate values includes 2 grams, 10 grams, and all intermediate values”). The endpoints of the ranges and any values disclosed herein are not limited to the precise range or value; they are sufficiently imprecise to include values approximating these ranges and/or values. All ranges are combinable.

As used herein, approximating language can be applied to modify any quantitative representation that may vary without resulting in a change in the basic function to which it is related. Accordingly, a value modified by a term or terms, such as “about” and “substantially,” may not be limited to the precise value specified, in some cases. In at least some instances, the approximating language can correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” can refer to plus or minus 10% of the indicated number. For example, “about 10%” can indicate a range of 9% to 11%, and “about 1” can mean from 0.9-1.1. Other meanings of “about” can be apparent from the context, such as rounding off, so, for example “about 1” can also mean from 0.5 to 1.4. Further, the term “comprising” should be understood as having its open-ended meaning of “including,” but the term also includes the closed meaning of the term “consisting.” For example, a composition that comprises components A and B can be a composition that includes A, B, and other components, but can also be a composition made of A and B only. Any documents cited herein are incorporated by reference in their entireties for any and all purposes.

### Overview of Example Embodiments

As a non-limiting example, a mechano-responsive patches can comprise of two aligned polycaprolactone (PCL)-polyethylene oxide (PEO) nanofibrous scaffold layers, fabricated via electrospinning, that are melt-stamped together using custom 3D-printed metal stamps. MAMCs are added in between nanofibrous layers before stamping, enabling their confinement inside the patches. The aligned nanofibers mimic the extracellular matrix microenvironment, providing topographical cues that enable cells to sense the material and infiltrate it for enhanced tissue integration and healing.

In order to assess the effects of the stamp design on the transfer of mechanical loads, three different stamp patterns were utilized, in which the longest length of the stamp rhombus pattern was varied from 3 to 5 mm. Different patterns enabled different levels of MAMC mechano-activation under dynamic tensile loads, indicating that modulating stamp designs can enable the tunability of MAMC activation to control the provision of the encapsulated drug. Greater loading cycles led to increased activation of embedded MAMCs.

To test the ability of the scaffold to be delivered and retained in vivo, scaffolds were implanted in goats to repair cervical disc injuries. The MAMC-laden mechano-responsive scaffolds were sutured over intervertebral disc injuries across the annulus fibrosus of the disc—a fibrous tissue that sustains mechanical loading during daily activity. The scaffold was attached over disc herniation injuries for disc repair and restoration of the tissue's mechanical function. After 4 weeks in vivo, the scaffolds were retained at the site of injury. Histological analysis of the scaffolds revealed robust cellular infiltration and extracellular matrix deposition around and throughout the scaffold, which enabled robust integration with the native tissue. We are currently awaiting samples from bioactive patch groups in which an anti-inflammatory drug was delivered to the injury site. Our proof-of-concept in vitro analyses and in vivo implantation study demonstrate that the mechano-responsive patch is able to delivery drug-encapsulating depots to the site of implantation, enabling localized drug delivery in response to time in vivo (degradation) and mechanical inputs (tissue loading environment). This represents a novel therapeutic platform that can be used to encapsulate and deliver a variety and/or combination of therapeutic molecules in a localized manner in tissues that receive mechanical inputs. Collectively, this study has shown that this programmable mechano-responsive therapeutic delivery approach can be expanded for application in other load-bearing environments to provide diverse therapeutic agents needed at different stages of repair for the treatment of a wide range of disorders.

### Illustrative Disclosure—I

The management of intervertebral disc (IVD) herniations through microdiscectomy can effectively alleviate symptoms. However, the injury through which the herniation occurs, including the torn annulus fibrosus (AF), is left unrepaired. This provides an uninterrupted path for re-herniation, aberrant scar infiltration, and nociceptive nerve ingrowth. The proinflammatory cytokine interleukin-1β (IL-1β) is present in herniated discs and contributes to a loss of extracellular matrix and increased nerve ingrowth by increasing the production of nerve growth factor (NGF) by disc cells. Thus, blocking IL-1β and NGF can prolong matrix retention and prevent nerve ingrowth. The purpose of this study was to (1) develop and optimize a mechano-responsive AF repair scaffold that delivers IL-1β and NGF blocking agents via mechanically activated micro-capsules (MAMCs) for local delivery and (2) assess the delivery, retention, and performance of the AF repair device in a goat cervical spine herniation model. We hypothesize that anti-IL-1β and anti-NGF encapsulating MAMCs can be integrated into an AF repair patch to provide molecule release, and that the annular repair patch can be delivered for repair of disc herniations in vivo in a large animal model.

### Methods

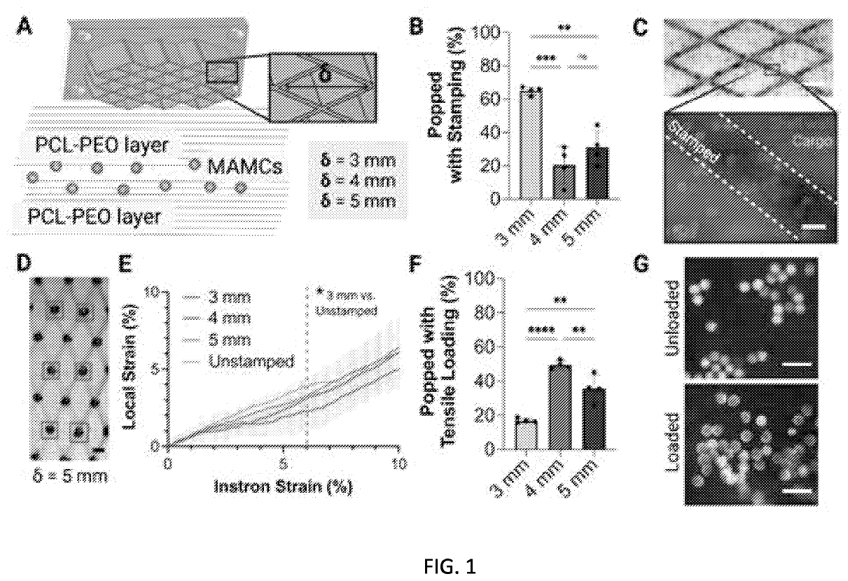

Patch Fabrication: Aligned polycaprolactone (PCL)-polyethylene oxide (PEO) nanofibrous scaffolds were fabricated via electrospinning. MAMCs were fabricated to encapsulate a model drug (bovine serum albumin, BSA), the IL-1 receptor antagonist, Anakinra (Sobi), or the anti-human NGF antibody, Tanezumab (Creative Biolabs). Annular repair devices were fabricated by melt-stamping MAMCs between two scaffold sheets (FIG. 1A). 3D printed rhombus-patterned metal stamps were produced in which the long diagonal of the rhombus (δ) was varied (3-5 mm).

Strain Tracking: Melt-stamped scaffolds were loaded in uniaxial tension along the fiber direction and optical strain tracking was performed up to 10% strain (FIG. 1D).

MAMC Activation with Stamping/Loading: After stamping, scaffold layers were separated and imaged (ZEISS Axio Zoom) to quantify popped MAMCs. To assess MAMC mechano-activation, scaffolds were subjected to dynamic tensile loading (6% applied strain, 1 Hz) for 1 hr.

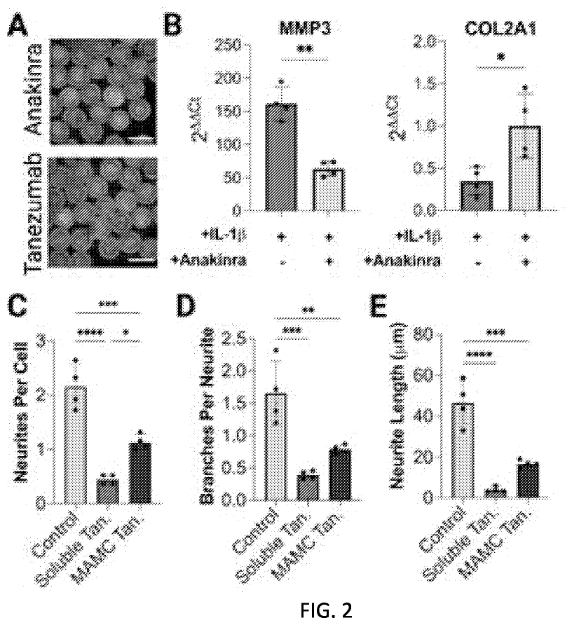

Drug Bioactivity Assessments: To test IL-1β-blocking effects of Anakinra, human AF cells from healthy donors (Articular Eng.) were cultured for 3 days±IL-1β (10 ng/mL)±Anakinra (100 ng/ml). qPCR was performed after treatment to assess changes in gene expression associated with matrix production and degrading enzymes. To test the effects of Tanezumab on neurite outgrowth, PC-12 cells (ATCC) were cultured with NGF (100 ng/mL)±Tanezumab (1 mg/mL) or Tanezumab from MAMC contents (2 mg/mL) for 6 days, after which phase contrast images were obtained for neurite quantification (FIG. 2C-E).

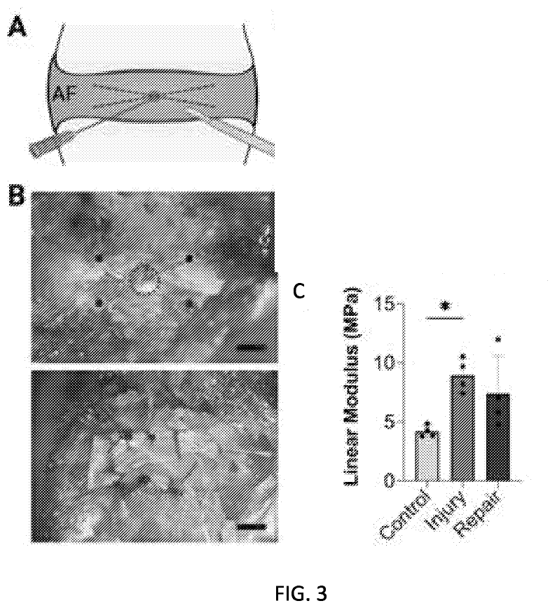

In Vivo Annular Repair Study: With IACUC approval, 4 female goats underwent annular injury and/or patch repair at C2-3 and C3-4, with C4-5 used as healthy controls. Annular injury was performed as in FIG. 3A, after which a BSA-MAMC control patch (l: 10 mm, w: 3.5 mm, δ=4) was sutured over the injury. Animals were euthanized at 4 weeks. Cervical spines were subjected to MRI at 3T for quantitative T2 mapping. Following MRI, motion segments were subjected to compressive mechanical testing (0 to −100 N, 0.24 MPa) to determine toe and linear moduli, transition, and maximum compressive strains. Motion segments are being decalcified and processed for paraffin histology. Statistical Analyses: Significant differences (*p<0.05, ** p<0.01, *** p<0.001, **** p<0.0001) were assessed via t-test or one-way ANOVA.

### Results

Stamp geometry impacted the number of MAMCs that popped with melt stamping, with δ=3 showing greatest rupture (FIG. 1B). Deltas of 4 and 5 mm resulted in strain transfer comparable to that of unstamped PCL (FIG. 1E). This led to greater activation of MAMCs with dynamic loading (FIG. 1F-G). MAMC fabrication enabled the encapsulation of Anakinra and Tanezumab (FIG. 2A). Anakinra treatment of AF cells in the presence of IL-1β decreased expression of MMP3 and increased the expression of COL2A1 (FIG. 2B). Tanezumab extracted from MAMCs effectively blocked the NGF-induced neurite outgrowth, similar to soluble Tanezumab delivery (FIG. 2C-E). Annular patches delivered to the goat cervical spine remained in place for 4 weeks. Average T2 maps showed reduced signal in the NP for the injury groups, compared to controls, which was improved towards control levels in the repair group. This was accompanied by an increase in linear modulus in the injury group, compared to healthy controls (FIG. 3C).

### Discussion

A mechano-active annular repair patch was developed to enable MAMC-mediated biomolecule delivery. Increasing δ preserved more MAMCs after stamping, preserved strain transfer, and consequently, increased activation of MAMCs under dynamic loading. Anakinra and Tanezumab were effectively encapsulated in MAMCs and attenuated catabolic responses in AF cells and limited neurite sprouting in PC-12 cells, respectively. Annular repair patches were successfully implanted in the goat cervical spine and remained in place over 4 weeks. While 4 weeks is not sufficient time to observe long-term degenerative changes, this early time point will capture the acute inflammatory changes that occur post-injury, including AF remodeling as observed via T2 quantification. Samples being processed for histology will reveal greater information on the microstructural, compositional, and cellular changes induced by injury and repair.

### Significance

Disc herniations lack effective repair approaches and surgical interventions remain palliative. This work represents the development of a bioactive annular repair device applicable to, e.g., repair of disc herniations and prevention of recurrent and or painful herniation. It should be understood, however, that although the disclosed technology is described using examples related to herniations, the disclosed technology is not limited to use with disc herniations and is suitable and useful for other applications.

### Illustrative Disclosure—II

Symptomatic intervertebral disc (IVD) herniations cause debilitating numbness and pain. While resection of the herniated tissue can alleviate these symptoms, the remaining compromised annulus fibrosus (AF) is left unrepaired, providing an uninterrupted path for aberrant scar infiltration, nociceptive nerve ingrowth, and recurrent herniation. The low AF cellularity, high density, and avascularity prevent endogenous healing and predispose the disc to degeneration.

Numerous approaches have attempted to seal the AF injury or deliver biologics to enhance repair. Although some of these approaches have partially restored IVD biomechanics, the inflammation-mediated loss of extracellular matrix (ECM), that exacerbates degeneration, has not yet been addressed. Therefore, there is a need for an annular repair device that provides both physical reinforcement and closure of the AF tear while simultaneously mitigating inflammation to prevent the degenerative loss of ECM.

Upon herniation of the disc, local expression of the proinflammatory cytokine interleukin-1β (IL-1β) increases, contributing to the acute loss of ECM post-injury. Elevated IL-1β also causes an increase in nerve growth factor (NGF)—a chemotactic signal that increases the infiltration of nociceptive neurites into the disc. Thus, blocking IL-1β signaling can prevent the inflammation-mediated loss of ECM and prevent recurrent pain.

Mechanically-activated microcapsules (MAMCs) are drug-encapsulating depots that have tunable mechano-activation and degradation properties, thus representing an ideal localized drug delivery system for the load-bearing IVD. The goals of this study were to (1) develop and optimize a mechano-responsive annular repair scaffold and (2) assess the delivery, retention, and performance of the AF repair device in a goat cervical spine herniation model. We hypothesized that repairing the torn AF post-herniation and blocking local IL-1β signaling would prevent the loss of AF ECM, decrease neurite and scar infiltration, and improve IVD biomechanics.

### Method

MAMC Fabrication: MAMCs were fabricated to encapsulate a model drug (bovine serum albumin, BSA) or the IL-1 receptor antagonist, Anakinra (Sobi).

Drug Bioactivity Assessments: To test the effects of increasing Anakinra concentrations on cell viability, Alamar Blue cell viability assay was performed on AF cells from healthy human donors (Articular Eng.) after 3 days of culture with soluble Anakinra. To assess drug bioactivity after encapsulation in MAMCs, the contents of Anakinra-loaded MAMCs or soluble Anakinra was added to human AF cells±IL-1β (10 ng/mL) for 3 days. qPCR was performed after treatment to assess changes in catabolic gene expression.

Patch Fabrication: Aligned polycaprolactone (PCL)-polyethylene oxide (PEO) nanofibrous scaffolds were fabricated via electrospinning. Annular repair devices were fabricated by heating up 3D-printed metal stamps to 80° C. and melt-stamping MAMCs between two scaffold sheets. Metal stamps with varied lengths of the longest rhombus diagonal (8:3-5 mm) were tested.

Scaffold Tensile Loading: Dynamic uniaxial tension cycles (6% strain, 1 Hz) were applied to MAMC-laden scaffolds along the fiber direction using a custom-built bioreactor, after which MAMC imaging was performed to quantify MAMC mechano-activation. To determine scaffold mechanical properties, scaffolds were subjected to uniaxial tension to failure with optical strain tracking of local deformation.

In Vivo Annular Repair: With IACUC approval, 8 goats underwent annular injury and/or patch repair at C2-3 and C3-4, with the C4-5 level used as a healthy control. Annular injury consisting of a partial-thickness AF laceration followed by full thickness needle puncture was performed. Scaffold groups received either a BSA-MAMC control patch (repair) (l: 10 mm, w: 3.5 mm, δ=4) or an Anakinra-loaded patch (Anakinra) that was sutured over the injury. Animals were euthanized at 4 weeks. Cervical spines were subjected to MRI at 3T for quantitative T2 mapping. Following MRI, motion segments were subjected to compressive testing (0 to −100 N, 0.24 MPa) to determine biomechanical properties. Motion segments were then fixed, decalcified, and processed for paraffin histology. Mallory-Heindenhain staining was performed, and collagenous infiltration was quantified using ImageJ.

Statistical Analyses: Significant differences were assessed via parametric or non-parametric one- or two-way ANOVA (* p≤0.05, **≤0.01, ***≤0.001, ***** 0.0001).

### Results

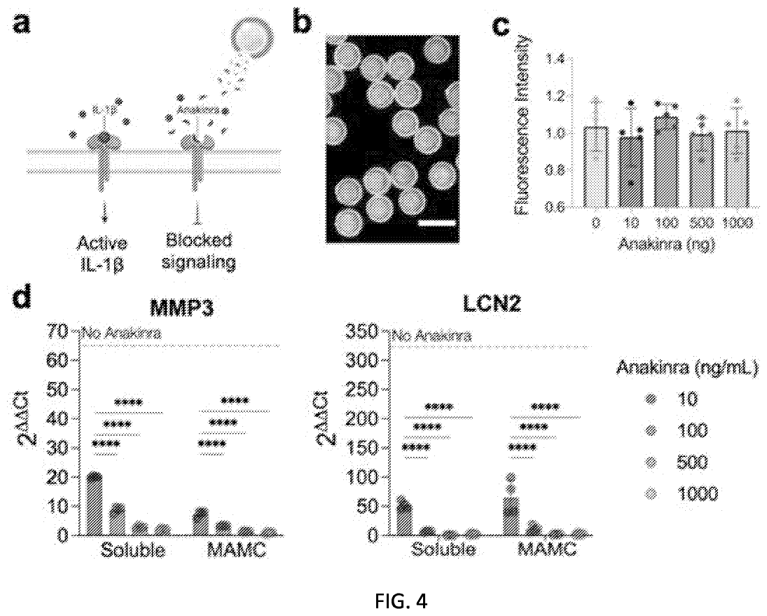

Anakinra-loaded MAMCs were effectively fabricated (FIG. 4a-4b). Anakinra concentrations of up to 1000 ng/mL did not have a detrimental effect on AF cell viability (FIG. 4c) and caused a concentration-dependent decrease in the expression of MMP3 and LCN2. Furthermore, the contents retrieved from Anakinra-loaded MAMCs demonstrated a comparable effect to that of soluble Anakinra (FIG. 4d).

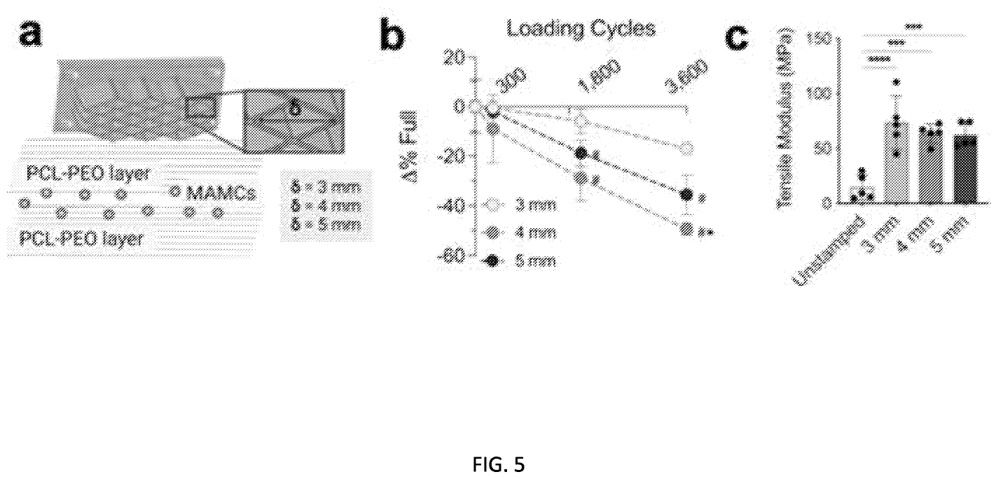

Annular repair scaffolds were formed by melt-stamping MAMCs between two fibrous scaffold layers (FIG. 5a). Dynamic tensile loading of scaffolds resulted in increasing mechano-activation of MAMCs with increasing number of loading cycles (FIGS. 5b). δ=4 and 5 mm stamp patterns created comparable MAMC mechano-activation profiles while the δ=3 pattern resulted in less activation with loading. Melt stamping increased the tensile modulus of the scaffolds compared to an unstamped scaffold of comparable thickness (FIG. 5c).

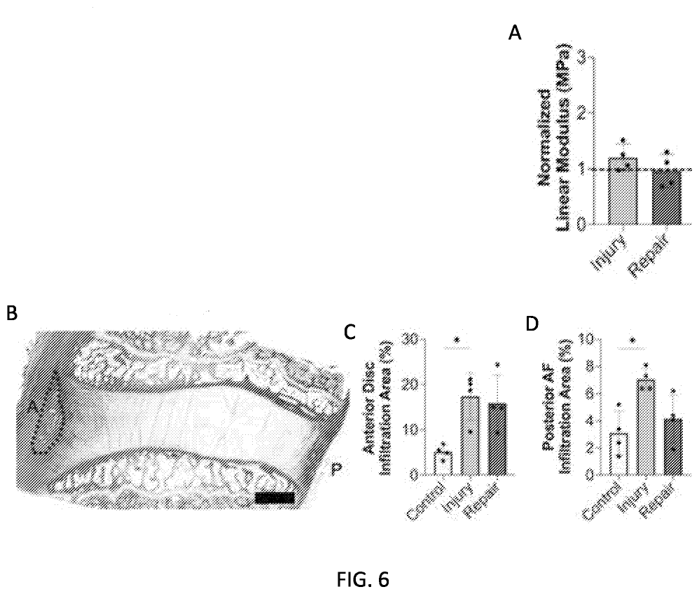

After 4 weeks in vivo, annular repair patches remained secured at the injury site. Repair with an empty patch maintained the disc linear modulus at uninjured levels, while stiffening occurred in the injury group (FIG. 6a). Empty patch delivery also reduced the collagenous infiltration observed on the anterior and posterior AF of injured discs (FIG. 6b-6d).

### Discussion

Anakinra potently blocked IL-1β signaling, attenuating the upregulation of catabolic genes in human AF cells in a dose-dependent manner. Therefore, the localized delivery of MAMCs with bioactive Anakinra can be used to provide therapeutically effective concentrations at the target injury site. MAMCs can be integrated into annular repair patches and mechano-activated in response to dynamic loading. The repair patches remained in place after 4 weeks in vivo. Interestingly, empty patch delivery prevented the stiffening of injury-only groups, potentially (though without being bound to any particular theory or embodiment) by decreasing the robust scar infiltration and collagenous remodeling observed after injury. While further analysis of Anakinra-loaded patch groups is in progress, the effects of Anakinra treatment in vitro and the T2 signal observed for the Anakinra patch group indicate that the simultaneous provision of structural reinforcement and anti-inflammatory molecules can prevent catabolic remodeling and loss of disc function post-herniation.

### Illustrative Disclosure—III

Conventional treatment for intervertebral disc herniation alleviates pain but does not repair the annulus fibrosus (AF), resulting in a high incidence of recurrent herniation and persistent disfunction. The lack of repair and the acute inflammation that arise after injury further compromises the disc and can result in disc-wide degeneration in the long term. To address this clinical need, we developed tension-activated repair patches (TARPs) for annular repair and the local delivery of bioactive anti-inflammatory factors. TARPs transmit physiologic strains to mechanically-activated microcapsules (MAMCs) embedded within, which activate and release encapsulated biomolecules in response to physiologic loading. Here, we demonstrate that the TARP design modulates implant biomechanical properties and regulates MAMC mechano-activation. Next, the FDA-approved anti-inflammatory molecule, interleukin 1 receptor antagonist, Anakinra, was loaded in TARPs and the effects of TARP-mediated annular repair and Anakinra delivery was evaluated in a model of annular injury in the goat cervical spine. TARPs showed robust integration with the native tissue and provided structural reinforcement at the injury site that prevented disc-wide aberrant remodeling resulting from AF detensioning. The delivery of Anakinra via TARP implantation improved the retention of disc biochemical composition through increased matrix deposition and retention at the site of annular injury. Anakinra delivery additionally attenuated the inflammatory response associated with scaffold implantation, decreasing osteolysis in adjacent vertebrae and preserving disc cellularity and matrix organization throughout the AF. These results demonstrate the applicability of the disclosed novel TARP system, e.g., for the treatment of intervertebral disc herniations.

The intervertebral disc (IVD) is a soft tissue that bridges adjacent vertebra throughout the length of the spine, providing flexibility, load transfer, and shock absorption during activities of daily living. In order to withstand the complex loads acting on the spine, the IVD has a unique configuration with regional variations in biochemical composition and tissue architecture. The disc core is a gel-like substance, termed the nucleus pulposus (NP), that has high proteoglycan and water content to withstand compressive forces. Surrounding the NP, the highly organized annulus fibrosus (AF) consists of aligned circumferential layers that enable the pressurization of the NP during compression while withstanding tension during flexion, rotation, and lateral bending. Connecting the NP/AF structure to superior and inferior vertebra are the cartilaginous endplates (CEPs). These substructures and the adjacent vertebra form the spinal motion segments which sustain and transmit physical loads during body movement.

Injuries to the disc resulting from trauma, overuse, or degeneration can cause annular ruptures and clefts. Tears that traverse the full AF thickness give way to extrusion of the NP and other disc tissue past the outer periphery of the disc. This type of injury, termed disc herniation, can result in compression of surrounding spinal nerves and chemical irritation due to elevated inflammatory cytokines, resulting in numbness and pain along the back and extremities.

Although not every disc herniation results in pain, symptomatic disc herniations are prevalent, affecting 2-3% of the population (5). The current gold standard for surgical management of symptomatic disc herniations is microdiscectomy, in which herniated tissue is surgically excised. Although this results in nerve decompression, the underlying disc pathology is not addressed, and results in an uninhibited conduit for additional NP extrusion and a persistent compromised AF structure with impaired biomechanical function. The lack of repair, coupled with the disc's inability to heal, makes recurrent disc herniations common, with reports of up to 25% in cases of lumbar disc herniation and predisposes the disc to continued degenerative changes. Therefore, there is a large unmet clinical need for new repair approaches that provide annular closure and help restore disc function.

Herniated tissue causes an inflammatory response, characterized by infiltration of macrophages and an upregulation in the production of inflammatory cytokines by disc cells and leukocytes. Importantly, the expression of the inflammatory cytokine interleukin 1β (IL-1β), increases with injury, aging, and degeneration. IL-1β increases the expression of matrix degrading enzymes and chemokines that recruit immune cells, establishing positive inflammatory feedback loops regulated by local concentrations of the cytokine. In addition, IL-1β upregulates production of neurotrophins, signaling molecules involved in the survival, differentiation, and migration of neurons. This ingrowth of nerves may play an integral role in the onset of back pain and hyperalgesia following disc injury or degeneration. Hampering IL-1β-mediated catabolic signaling after annular injury might prevent further loss of extracellular matrix, decrease back pain, and improve tissue repair, and thus represents a promising therapeutic avenue for the treatment of disc herniations.

To that end, we developed a tension-activated repair patch (TARP) that acts to both provide physical closure of the ruptured disc and deliver bioactive agents to the site of injury in response to mechanical loading in the local tissue microenvironment. The base unit of the TARP is a layer of composite electrospun scaffold, designed to mimic the architecture of the AF while promoting rapid cellular infiltration. These layers are assembled into the TARP via thermal stamping to include pockets containing mechanically-activated microcapsules (MAMCs) that activate in response to local deformation. We investigated the effects of TARP design on scaffold biomechanics, MAMC mechano-activation, and cellular organization. TARP stamping pattern dictated scaffold biomechanics and the transfer of local strains, impacting the mechano-activation of MAMCs under physiologic dynamic loading. To block IL-1β signaling, the FDA-approved interleukin 1 receptor antagonist, Anakinra (Sobi), was encapsulated within the MAMCs and these were integrated into the TARPs. The effects of TARP repair and TARP-mediated Anakinra delivery on the repair of annular injuries was assessed in a goat cervical disc injury model. After four weeks, TARPs remained in place at the site of implantation and integrated with the native tissue, as evidenced by robust extracellular matrix deposition throughout the TARPs. The structural support provided by TARP implantation prevented aberrant remodeling of the disc associated with AF de-tensioning post-injury, as shown by a retention of cellularity along the posterior AF and reduced necrosis and collagenous remodeling. TARP-mediated Anakinra delivery at the site of injury also improved the infiltration of repair tissue and the closure of annular injuries, which consequently preserved the normal demarcation of the NP/AF border and proteoglycan staining intensity in the NP. These results highlight the utility of the disclosed repair systems.

### TARP Stamp Pattern Affects MAMC Patency and Scaffold Mechanical Properties

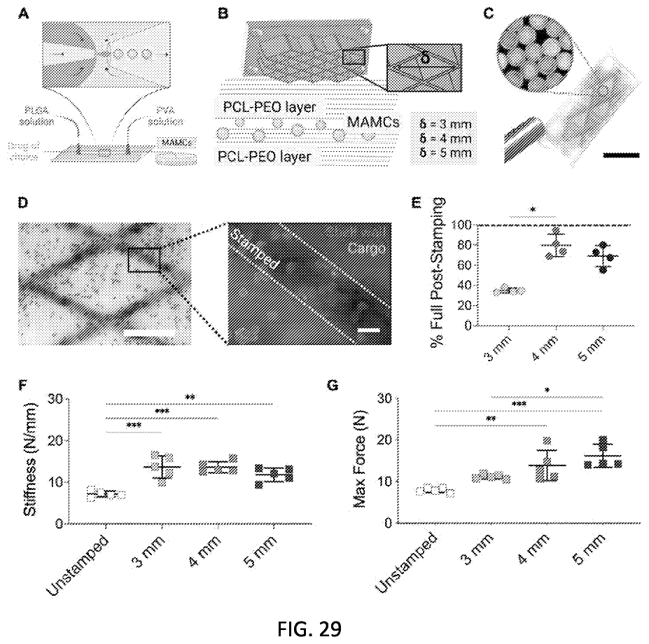

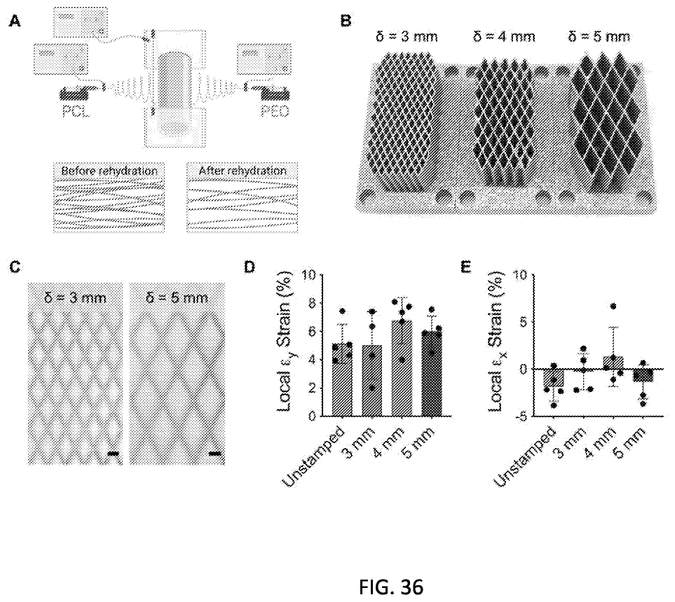

MAMCs and PCL-PEO nanofibrous scaffolds were fabricated (FIG. 29A, FIG. 36A). TARPs were assembled by melt stamping MAMCs between two PCL-PEO nanofibrous scaffolds (FIG. 29B-C). Melt stamping was performed using metal stamps that were preheated to 80° C., a temperature that, when combined with pressure, adhered the scaffold layers to form a robust multi-part assembly that could be peeled apart for visualization and analysis of encapsulated MAMCs (FIG. 36B-C). The combination of high temperature and applied pressure in the regions in which the stamps met the scaffold-MAMC-scaffold caused a loss of MAMC contents due to melting and/or crushing (FIG. 29D). This loss of MAMC contents depended on the stamp geometry. Specifically, varying the longest rhombus diagonal 8 caused significant changes in MAMC patency upon stamping (FIG. 29E). The 3 mm δ pattern showed the greatest loss of MAMC contents upon stamping, eliciting a loss of contents in approximately 60% of MAMCs. Conversely, stamping with the 4 and 5 mm δ patterns retained the contents of a greater proportion of MAMCs, with only ˜20% and 30% of MAMCs, respectively, losing their cargo (FIG. 29E). Ruptured MAMCs were localized to areas that came in direct contact with the stamp (FIG. 29D), indicating that the loss of cargo induced by the 3 mm δ stamp occurred due to a greater contact area during stamping.

To determine how different stamp pattern geometries impacted TARP biomechanical properties, unstamped scaffolds or TARPs were subjected to a tensile ramp to failure test, and several properties were assessed. TARP biomechanical properties changed with stamping, with differences related to the rhomboid parameter δ. Compared to unstamped scaffolds of comparable thickness, stamped TARPs had greater stiffness and maximum force (FIG. 29F-G), indicating that adhering the PCL-PEO layers via melt-stamping generated a composite structure with greater tensile strength than a single PCL-PEO layer. Increasing δ resulted in incremental increases in maximum force (FIG. 29G). TARP biomechanical properties could therefore be modulated via variations in stamp geometry.

### TARP Rhombus Dimensions can Influence MAMC Mechano-Activation

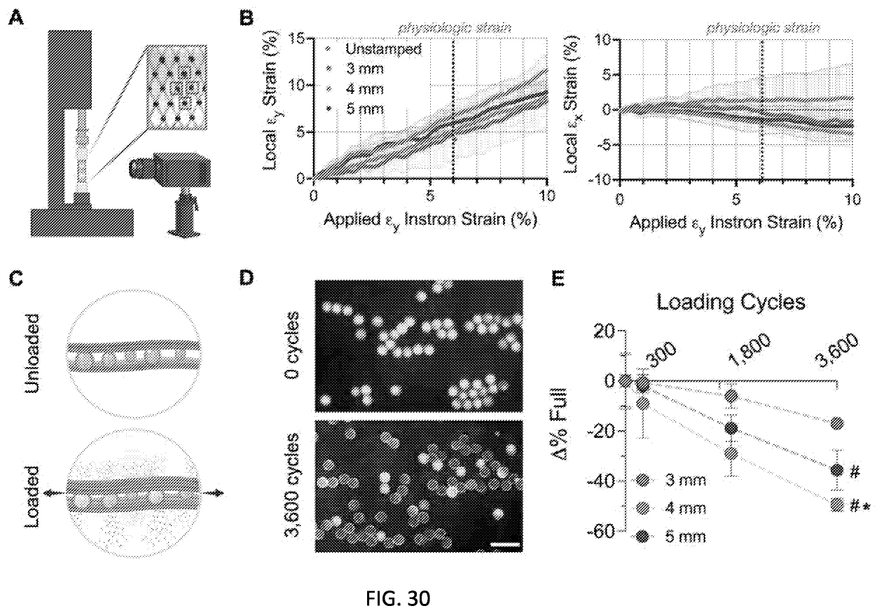

To determine the role of different stamp geometries on the transfer of strain, local strains (i.e., inside each stamped rhombus) were measured using optical tracking of marked rhombus vertices during the application of uniaxial tension (FIG. 30A). Tension was applied along the direction of fiber alignment, which was also the direction of the longest rhombus diagonal. Comparisons between measured longitudinal local strains (ε_{y}) and applied strain up to 10% revealed differences caused by variations in stamp δ (FIG. 30B). No significant differences were observed at physiologic outer AF strain levels (applied strain of 6%), though the mean local ε_{y} for the δ=4 mm group was higher than that of the unstamped scaffolds (FIG. 36D). This trend was apparent up to the 10% applied strain. A stamp pattern of δ=5 mm also showed ε_{y} levels above those measured on unstamped scaffolds, though to a lesser extent than for the δ=4 mm pattern. Conversely, a δ of 3 mm hindered the transfer of strain and led to longitudinal strains less than those measured on unstamped scaffolds. Melt stamping also altered the transverse strains (εₓ) measured across TARPs compared to unstamped scaffolds. While unstamped scaffolds had decreasing εₓ as ε_{y} increased, the transverse strains measured on δ=4 mm TARPs were minimal despite an increasing ε_{y}. δ=3 and 5 mm stamped scaffolds also showed an εₓ that was lower than the unstamped scaffold (FIG. 36E). Together, these results highlighted that, compared to unstamped scaffolds, TARPs with a stamp pattern of δ=4 mm were able to transfer greater longitudinal strains during tensile loading and that melt-stamping reduced the transfer of strain in the transverse direction.

Physiological tensile forces along the outer AF are dynamic in nature, occurring throughout daily locomotion and activity. To characterize the effects of the local strain transfer differences caused by melt stamping patterns on MAMC mechano-activation under physiologic loading (FIG. 30C), TARPs were assessed under dynamic tensile loading (6% strain, 1 Hz) using a custom motorized uniaxial bioreactor. The roles of stamp pattern and loading cycles on the mechano-activation of MAMCs were determined by measuring % Full MAMCs with and without loading (FIG. 30D). Increasing cycles of loading led to incremental mechano-activation of MAMCs, irrespective of stamping pattern. After 300 loading cycles, there were no differences in % Full compared to unloaded TARPs, regardless of stamp δ (FIG. 30E). However, after 1,800 and 3,600 cycles of loading, significant differences were observed between different TARP designs (FIG. 30D-E). Specifically, larger stamp δ patterns caused greater mechano-activation, with δ=4 and 5 mm showing increased levels of MAMC mechano-activation compared to the δ=3 mm TARPs. Interestingly, the 4 mm δ TARPs showed the greatest mechano-activation compared to other TARP stamp patterns (FIG. 30D). After 3,600 cycles, ˜50% of MAMCs mechano-activated in the δ=4 mm TARPs while ˜38% and 18% of MAMCs ruptured in the 5 mm and 3 mm δ TARPs. The increased mechano-activation of MAMCs for the 4 mm δ stamp design coincided with the greatest strain transfer measured in the longitudinal direction (and least transverse strain), suggesting that the strain transfer regulated by stamp design directly impacted the mechano-activation of TARP-encapsulated MAMCs.

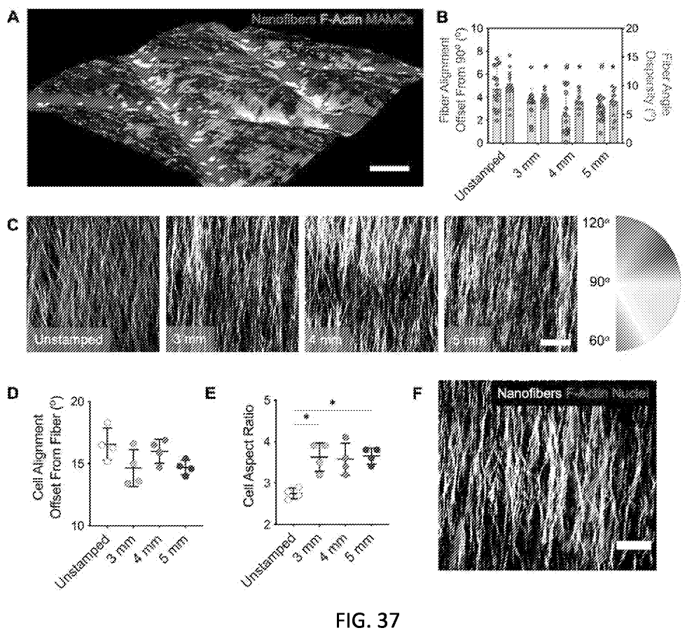

The process of melt stamping generates curvature emanating from the points of melt stamping in which pressure and increased temperature anneal the scaffold layers together (FIG. 37A). To understand how this curvature affected the alignment within the scaffold layers, fibers were visualized before and after melt stamping and alignment was measured. Melt stamping increased fiber alignment, observed through a decrease in the fiber offset from 90° (FIG. 37B-C) after stamping. Larger rhombus δ (4 and 5 mm) patterns significantly increased fiber alignment compared to unstamped scaffolds. This was accompanied by a reduction in fiber angle dispersity—a measure of the heterogeneity or spread in fiber alignment-regardless of stamp δ (FIG. 37B-C). These results indicate that melt stamping increased fiber alignment and reduced fiber dispersity, with greater fiber realignment observed in TARPs created with larger stamp δ.

Nanofibrous scaffolds present topographical cues through their nano-architecture and organization that affect cellular sensing, and ultimately, cellular morphology. To assess the effect of changes in fiber alignment on cellular morphology, human AF cells were cultured on unstamped scaffolds or on TARPs of different stamp δ. After 6 days of culture on each material, cells were stained and visualized. As expected, cells showed increased alignment with melt stamping, which corroborated the increased fiber alignment (FIG. 37D). This increase in cellular organization was accompanied by an increased cell aspect ratio on melt stamped TARPs, with cells demonstrating highly extended cell bodies along the fibers (FIG. 37E-F). Melt stamping therefore causes a structural reorganization of nanofibers, resulting in increases in fiber and cellular alignment with stamping, impacting cellular interactions.

### MAMC-mediated Delivery of Anakinra Attenuates Catabolic Gene Expression in AF Cells

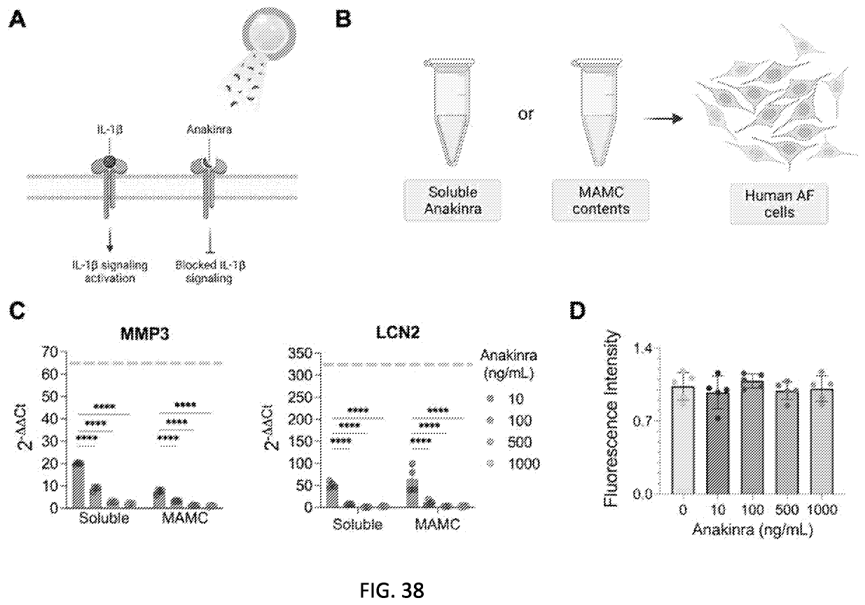

The FDA-approved interleukin 1 receptor antagonist, Anakinra, has potent IL-1β inhibitory effects, including when delivered from MAMCs. To verify that MAMC-mediated Anakinra delivery effectively inhibited IL-1β signaling, human AF cells were cultured in the presence of IL-1β and then treated with Anakinra delivered directly into the media (soluble) or extracted from MAMC contents before media supplementation (FIG. 38A-B). Gene expression analysis for the catabolic genes MMP3 and LCN2 demonstrated a significant upregulation when the cells were exposed to IL-1β without the addition of Anakinra. This upregulation was drastically reduced in a dose-dependent manner with the addition of Anakinra, both in the soluble and MAMC-extracted form (FIG. 38C). Assessments of cell viability verified that Anakinra concentrations of up to 1,000 ng/ml did not have a detrimental effect on AF cell survival (FIG. 38D). Together, these results demonstrated the potential of Anakinra provision to attenuate IL-1β-mediated catabolic signaling in AF cells.

### TARP-Mediated Delivery of Anakinra in a Goat Cervical Spine Disc Herniation Model

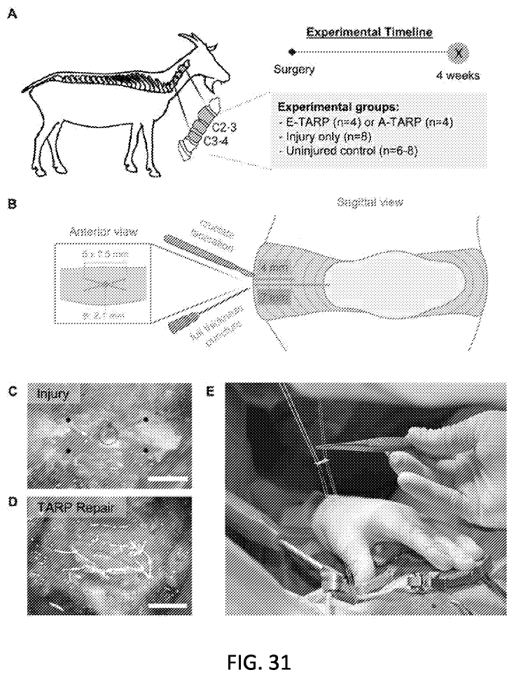

To determine if TARPs can aid in the repair of annular tears with or without the delivery of an anti-inflammatory molecule (IL-1ra, Anakinra), empty TARPs and Anakinra-loaded TARPs were implanted in vivo in a large animal cervical disc annular injury model for up to 4 weeks. Due to the increased retention of MAMC contents post-stamping, higher strain transfer, and MAMC mechano-activation under dynamic tensile loading observed for the 4 mm δ TARPs, this stamp geometry was chosen for this in vivo study. TARPs were assembled with dimensions previously determined to fit within the goat cervical disc space, loaded with MAMCs containing a BSA model drug (E-scaffold) or Anakinra (A-scaffold). A total of n=8 goats underwent surgery, where the C2-3 and C3-4 cervical discs received annular injury followed by TARP repair of one injured level per animal (FIG. 31A-B). Adjacent levels were used as uninjured controls. The annular injury consisted of a partial thickness X-shaped laceration on the anterior AF followed by a full thickness needle puncture, mimicking the full-thickness tears that give way to NP leakage and subsequent disc herniation (FIG. 31B-C). Levels that underwent TARP repair received either E-scaffolds or A-scaffolds, which were sutured in place over the annular injuries (FIG. 31D-E). There were no surgical complications arising from the disc injuries or the TARP implantation, and all animals recovered uneventfully until the study end date.

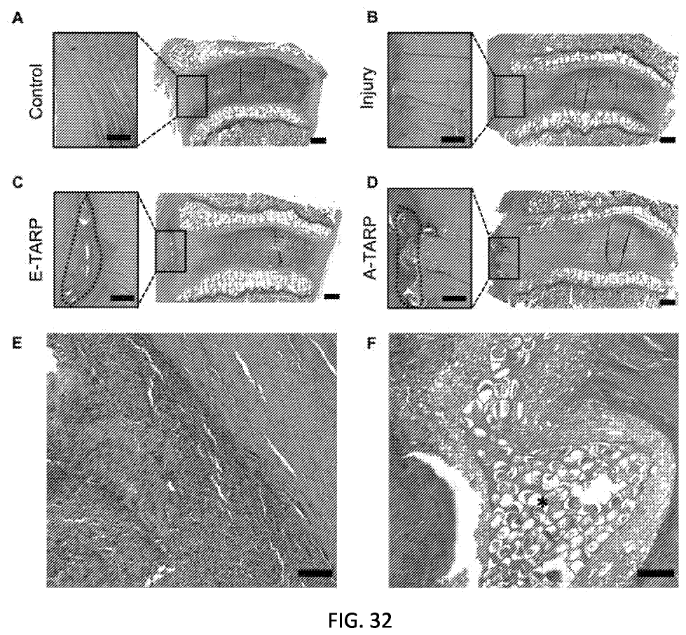

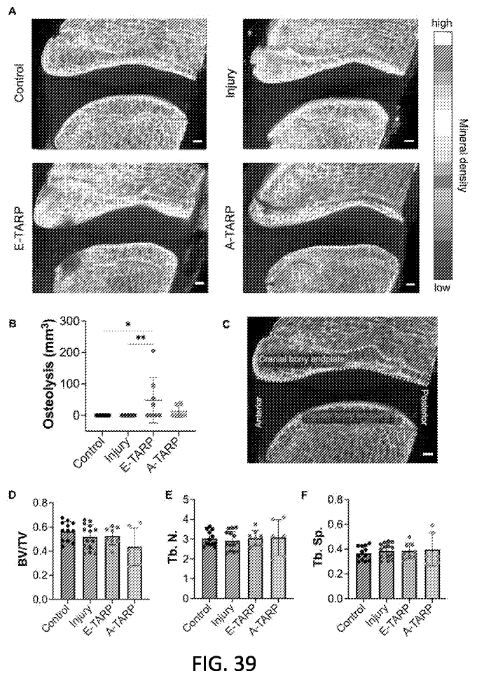

TARPs remained securely attached at the site of implantation after 4 weeks. Integration of the TARPs with the native tissue was observed through H&E staining of histological sections (FIG. 32), which showed robust deposition of extracellular matrix next to and throughout the expanse of each TARP (FIG. 32C-F). MAMC outlines were still visible after 4 weeks (FIG. 32F). MicroCT analysis revealed that E-Scaffold-treated levels had osteolysis along the anterior aspect of the adjacent vertebras while discs that only received injury did not show signs of bone loss (FIG. 39A). Interestingly, Anakinra-loaded TARPs did not elicit significant osteolysis and were comparable to uninjured control levels (FIG. 39B), suggesting that Anakinra delivery attenuated the inflammation-mediated loss of bone caused by TARP implantation. To determine if other changes in bone microarchitecture occurred as a result of injury or TARP repair, the cranial and caudal bony endplates adjacent to the discs were contoured and evaluated by microCT. Bone morphometric analyses revealed no differences between groups in bone volume/total volume, trabecular number, and trabecular spacing (FIG. 39C-F).

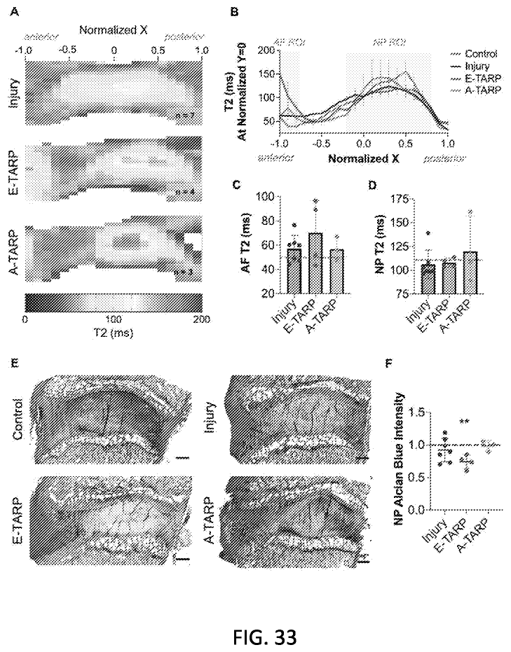

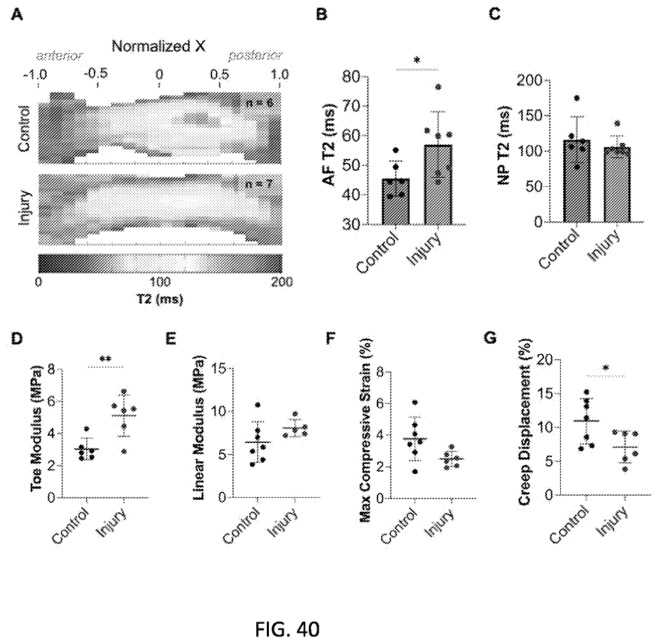

T2-weighted magnetic resonance imaging (MRI) is commonly used to assess disc health and NP T2 relaxation times correlate with disc hydration and proteoglycan content. Average T2 maps for each group revealed differences in AF T2 and NP/AF border morphology (FIG. 33A-B, FIG. 40A). In uninjured discs, NP T2 signal is centrally condensed and surrounded by a lower AF T2 signal. With injury, this pattern spread laterally towards the anterior and posterior AF (FIG. 33B, FIG. 40A). This resulted in a significant increase in AF T2 (FIG. 40B), measured at the anterior AF, while NP T2 was unchanged (FIG. 40C). Repair of injured discs with TARPs caused a greater retention of the NP/AF border compared to injured discs (FIG. 33A-B), suggestive of a faster closure of the injury tract that may have prevented NP leakage or spreading. While repair with E-scaffold increased the anterior AF T2, annular repair with the A-scaffold did not affect AF T2 (FIG. 33C). This suggests that Anakinra delivery may have attenuated an inflammatory response induced by E-scaffold implantation, consistent with the reduced osteolysis findings noted above. There were no differences in NP T2 across all treatment groups (FIG. 33D) despite an apparent anterior shift of the NP with injury. Overall, this MRI data suggested that the TARPs improved the repair of the AF injury tract and prevented anterior spreading of the NP, and that Anakinra delivery attenuated inflammatory remodeling at the site of TARP implantation.

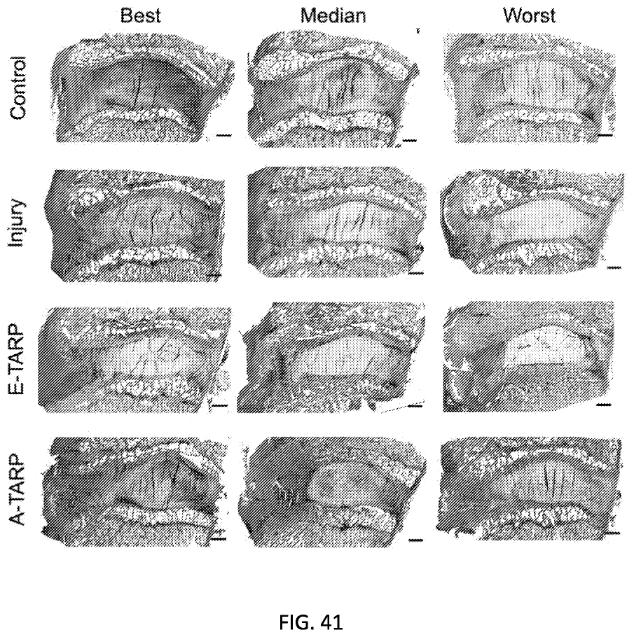

To determine if the differences in T2 relaxation times were caused by changes in biochemical composition, sagittal histological sections were co-stained with Alcian Blue and Picrosirius Red to visualize proteoglycans and collagens, respectively. Stark differences in NP proteoglycan staining intensity were observed between groups and this was verified through quantification of Alcian Blue staining intensity in the NP region of interest (FIG. 33E-F, FIG. 41). As expected, the highest proteoglycan staining intensity was measured in uninjured control discs (FIG. 33F). Injury caused a slight reduction in NP proteoglycan staining intensity compared to controls, albeit not statistically significant. E-Scaffold-treated discs showed the greatest reduction in NP proteoglycan staining intensity and was significantly lower than uninjured controls (FIG. 33F). Interestingly, TARP-mediated Anakinra delivery prevented this loss of NP proteoglycans, resulting in proteoglycan staining comparable to that of uninjured discs (FIG. 33F). Intense NP proteoglycan staining was apparent across all A-Scaffold discs analyzed when compared to the best, median, and worst specimens for each group, selected based on NP Alcian Blue intensity (FIG. 41). This suggested that Anakinra delivery via TARP implantation helped retain disc biochemical composition after injury, in accordance with the MRI T2 data.

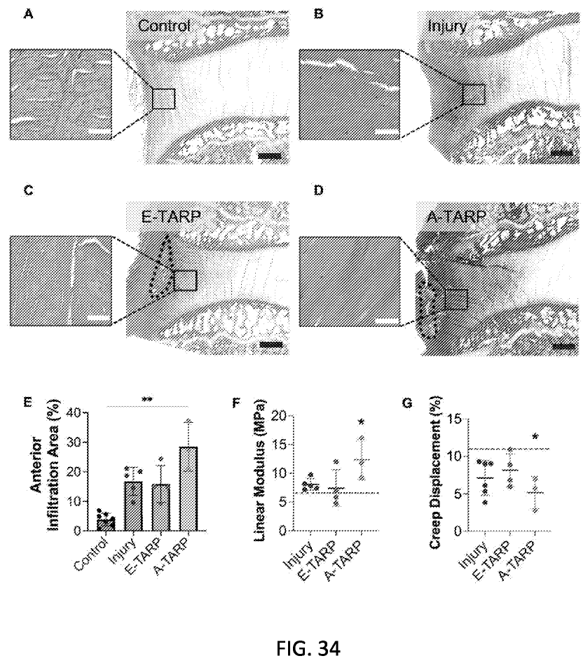

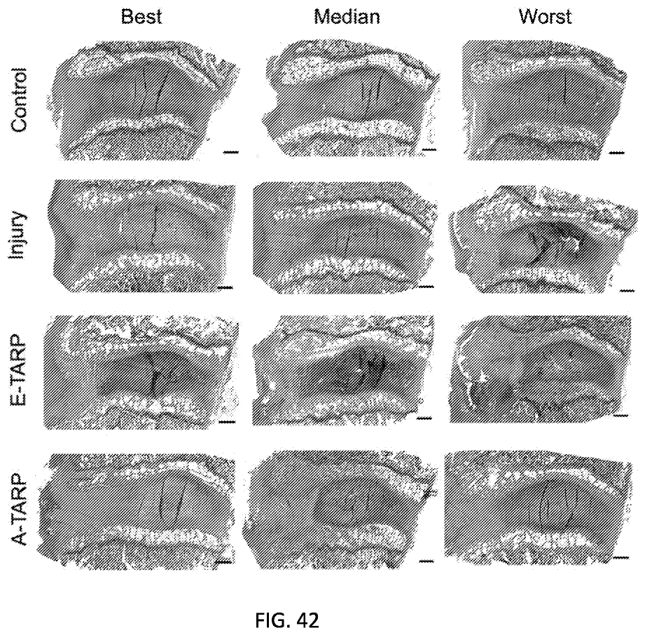

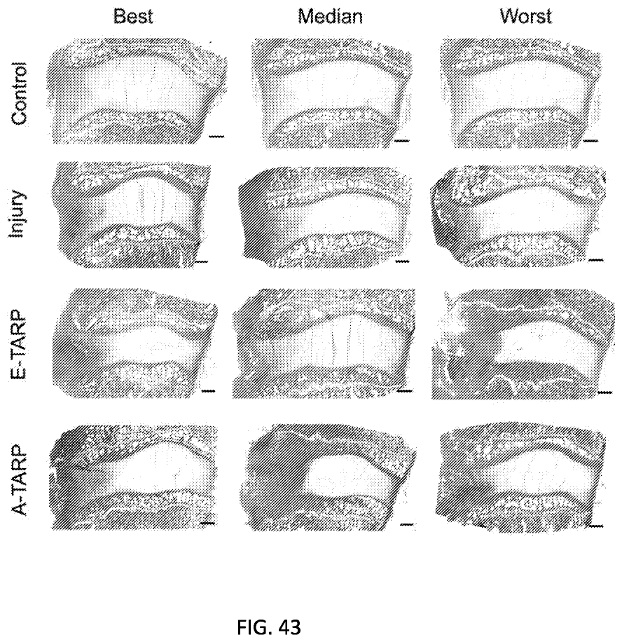

The large differences in proteoglycan staining intensity between E-Scaffold and A-Scaffold-treated discs suggested differences in healing response. To determine how these differences arose, we examined the injury location more closely. Sagittal histological sections were stained with hematoxylin and eosin (H&E) for visualization of cellular morphology and Mallory Heidenhain stain for inspection of scar infiltration and collagenous remodeling. Close inspection of the anterior AF revealed a loss of cellularity near the injury tract irrespective of treatment (FIG. 34A-D), suggesting that tissue injury induced necrosis and apoptosis near the site of insult, consistent with previous reports. Mallory Heidenhain staining revealed differences in scar tissue infiltration at the anterior AF (FIG. 34A-D), verified through quantification of scar tissue infiltration area (FIG. 34E). Granulation tissue composed mainly of collagen was stained with a dark purple color, showing infiltration of scar tissue along the outer third of the anterior AF for injury and E-Scaffold groups (FIG. 34A-E). In contrast, injury repair with Anakinra-loaded TARPs (A-Scaffold) resulted in greater infiltration and closure of the injury tract along the anterior AF (FIG. 34D-E). The greater infiltration of scar tissue and consequent closure of the annular injury may have prevented shifting or loss of NP material, resulting in greater retention of the NP/AF boundary (FIG. 33A) and NP proteoglycan content (FIG. 33E-F). This was supported by the differences in scar tissue infiltration and retention of the NP/AF border that was apparent in the H&E and Mallory Heidenhain-stained sections that represented best, median, and worst sections chosen based on NP Alcian Blue intensity (FIG. 42, FIG. 43).

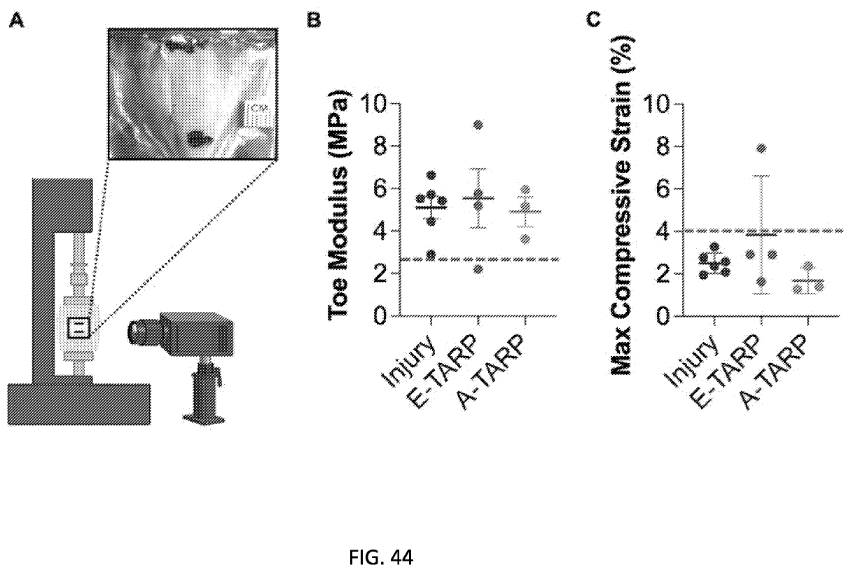

The extent of anterior scar infiltration affected disc biomechanics, determined through motion segment testing under cyclic compression and compressive creep at physiologic loads. Injured and E-Scaffold-treated discs, which had similar levels of anterior scar infiltration, showed comparable degrees of disc stiffening (FIG. 34E, FIG. 40D-G, FIG. 44). The levels repaired with A-Scaffolds showed greater stiffening with significant differences in linear modulus and creep displacement, compared to uninjured controls (FIG. 34E, FIG. 40D-G). This is suggestive of stiffening of the disc after 4 weeks of TARP-mediated Anakinra delivery and disc repair.

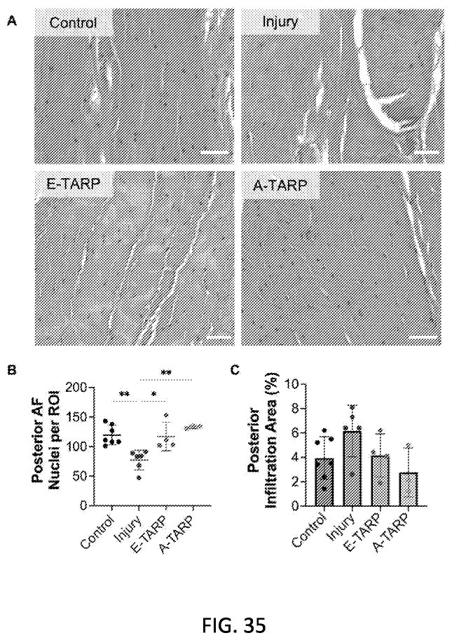

Annular lesions and herniation are related to degeneration of the surrounding tissue as a consequence of detensioning of the AF that causes disc-wide aberrant remodeling (24). For this reason, the posterior AF was inspected for signs of catabolic changes. Close analysis of H&E-stained sections along the posterior AF revealed dramatic differences in cellularity among groups (FIG. 35A). Quantification of cell nuclei across three regions of interest along the length of the posterior AF revealed a marked loss of cells, characteristic of tissue necrosis, along the posterior AF of injured discs that did not receive repair (FIG. 35B). In contrast, both E-Scaffold and A-Scaffold-treated discs retained cellularity levels comparable to uninjured controls, suggesting that TARP delivery provided mechanical reinforcement of the injured AF that prevented degenerative remodeling across the disc (FIG. 35B). Close inspection of Mallory Heidenhain-stained histological sections revealed differences in collagenous remodeling along the posterior region of the AF. Quantification of this posterior remodeling revealed an increase in collagenous remodeling with injury (FIG. 35C). Interestingly, TARP implantation, regardless of MAMC content, maintained posterior AF remodeling at uninjured control levels (FIG. 35F), in keeping with the posterior AF cellularity and necrosis findings. Together these results highlight that TARP implantation provides reinforcement of the AF, preventing disc-wide remodeling and tissue necrosis after injury.

### Discussion

The repair of AF injuries via an annular patch that restores the compromised disc structure and simultaneously delivers factors to aid in healing holds promise as a treatment approach for herniated intervertebral discs. There are currently no FDA-approved devices for the repair of the disc structure following herniation injury or the prolonged provision of biologic agents to the AF. A successful repair device must restore the combined function of all disc substructures to ensure its effective functioning as a single load-bearing unit. The device must also cause minimal damage to the native tissue upon implantation and must also provide reinforcement at the injury site to prevent degenerative changes that result from AF detensioning. Biologic supplementation of the avascular disc to attenuate inflammation at the injury site can further aid in tissue regeneration by limiting degradation of disc ECM and cellular apoptosis. For clinical translation, the repair device must demonstrate successful mechanical and biological support of IVDs of comparable anatomy, size, and mechanics to human discs. These challenges have impeded the translation of effective repair approaches to the clinic.

In this study, we developed and evaluated the tunable design of TARPs for closure of annular lesions and provision of anti-inflammatory molecules locally to the injury site. Through modifications of the TARP stamping pattern, biomechanical properties and rate of MAMC mechano-activation were varied, providing flexibility in the design of a repair device with a set of desired characteristics. The MAMC drug delivery system offers added tunability over drug provision, with the ability to choose MAMC rate of degradation and sensitivity to mechanical loading through the choice of PLGA employed and modification of microcapsule dimensions during fabrication. A single population or a combination of MAMC populations with different characteristics can therefore be used to provide delivery of a range of molecules at different times post-implantation. In this study, MAMCs were used to deliver the FDA-approved small molecule Anakinra, which effectively inhibited the upregulation of catabolic genes in AF cells cultured with IL-1β, demonstrating the promise of this therapeutic target for the prevention of matrix degeneration post-injury.

Building on these promising in vitro results, we translated empty or Anakinra-loaded TARPs for the repair of annular injuries in the goat cervical spine for up to 4 weeks. The goat cervical spine is an attractive preclinical model due to its semi-upright stature and its approximation of human cervical spine dimensions. Our results demonstrated robust retention, infiltration, and integration of the TARP implants with the native tissue. Implantation of TARPs alone, acting as a physical barrier, was sufficient to prevent tissue-wide degenerative changes through the provision of structural reinforcement of the compromised AF. TARP-mediated Anakinra provision at the repair site further improved the retention of the distinct sub-compartments of the disc, maintaining the NP/AF boundary by preventing NP displacement. Anakinra delivery also attenuated the inflammatory response associated with device implantation, which often can lead to device failure and further catabolic changes at the implantation site.

Due to the dense composition of the fibrocartilaginous AF, attachment of annular repair devices remains a challenge. The only FDA-approved device intended to physically block recurrent herniation is the Barricaid Annular Closure device (Intrinsic Therapeutics, Inc., Woburn, MA)—a bone-anchored implant with a polymer fabric end that is placed adjacent to the AF herniation. Although this device avoids damaging the AF during implantation, the attachment sites at the adjacent vertebras, where metal screws are inserted, have shown significant osteolysis in preclinical investigations. To avoid this issue, most work in the field thus far has been focused on the development of gel-based sealants or adhesives to seal AF lesions. Although adhesives provide ease of delivery via direct injection into the injury site, several have shown inadequate retention of disc mechanical function and have failed to prevent degenerative changes, possibly due to the lack of infiltration and deposition of matrix by endogenous cells. In this study, we demonstrated successful attachment of the TARPs to the AF using micro sutures, which enabled the retention of the TARPs at the implantation site after 4 weeks of unrestricted physical activity. The retention of the scaffold at the injury site permitted the cellular infiltration of TARPs by native cells that deposited dense matrix throughout the entirety of the scaffolds, which in turn ensured robust integration with the surrounding tissue. Although an effective method for scaffold attachment, manual suturing of the TARPs is not required, and methods that at least partially automate the suturing and provide greater ease of implantation can be used.

The physical attachment of TARPs over AF injuries prevented degenerative remodeling across the IVD structure. Under static equilibrium, the swelling pressures in the NP create residual strains in the AF that exceed 10% strain in the outer AF. The release of this residual strain by annular lesions and herniation accelerate the degeneration of the surrounding tissue by instigating aberrant remodeling, short-term apoptosis, and the adoption of atypical fibrotic cellular phenotypes. In our model of annular injury, where the anterior AF was disrupted, unrepaired levels showed aberrant fibrotic remodeling, AF necrosis, and cellular apoptosis along the posterior AF after 4 weeks. These aberrant changes were not present in cervical discs that received TARP implantation, indicating that the mechanical reinforcement of the AF provided by the repair scaffolds may reestablish residual strains and prevent disc-wide remodeling.

The delivery of Anakinra through TARP implantation also provided several benefits. Some of the main hallmarks of disc degeneration include the depletion of NP proteoglycan content and a loss of the NP/AF boundary. Compared to empty TARP groups, Anakinra-loaded TARPs demonstrated an improved retention of NP proteoglycan content and the NP/AF boundary. This was accompanied by an increased infiltration of scar tissue along the anterior AF, which provided larger closure of the annular lesion and prevented NP displacement through the annular tear. This deep scar infiltration contrasts the weak scar deposition observed in human specimens and animal models of disc herniation where scar infiltration is limited to the outer third of full thickness annular lesions. Anakinra provision also decreased osteolysis that was apparent with empty TARP delivery, indicating that blocking IL-1β signaling helped to attenuate the body's response to the foreign material. Therefore, blockade of IL-1β signaling local to the injury site represents a promising therapeutic target to supplement annular closure strategies for disc herniation management.

The TARP annular closure system has applicability in preclinical and clinical use. Although our study was limited in sample size and only assessed the short-term one-month timepoint, the clear signs of therapeutic benefits are seen. Molecules that can be used, e.g., for annular healing, include anti-apoptotic drugs and pro-anabolic agents. Different MAMC populations can be used, as well. The findings from this study show the value of the TARP system and demonstrate that simultaneous repair and provision of molecules to the AF for the treatment of disc herniations is feasible.

### Study Design

The objectives of this study were to develop tension-activated repair patches (TARPs) containing mechanically-activated microcapsules (MAMCs) and to elucidate the reparative effects of TARP-mediated annular repair and anti-inflammatory drug provision in vivo in a goat cervical disc injury model. We hypothesized that TARPs would provide structural reinforcement at the site of injury and enable anti-inflammatory drug delivery through MAMC mechano-activation, attenuating inflammation-induced matrix degradation.

TARPs were fabricated with different rhombus stamping patterns through variations in the longest diagonal of the rhombi (δ). The effects of stamp δ on MAMC patency after melt stamping was investigated (n=4/type), followed by characterization of TARP biomechanical properties (n=5/type). Local strain transfer under tension in response to stamp δ was investigated (n=5/type) and the effects of these differences on MAMC mechano-activation under increasing cycles of dynamic tensile loading at physiologic strains was investigated (n=4/type). To probe the effects of stamp design on fiber alignment and cellular morphology, fiber alignment and dispersity were measured post-stamping (n=4/type, n=4 ROIs/scaffold) and the morphology of human AF cells seeded on TARPs of different δ was assessed (n=4/type, n=30 ROIs/scaffold). To verify that MAMC fabrication did not alter inflammatory drug bioactivity, the interleukin 1 receptor antagonist drug, Anakinra, was encapsulated in MAMCs and its effect before and after encapsulation on the expression of catabolic genes by human AF cells, cultured in the presence of IL-1β, was assessed (n=4/group). Furthermore, a concentration sweep using different concentrations of Anakinra was performed to determine whether high drug concentrations affected AF cell viability (n=5/group). These studies enabled us to establish the most suitable stamping pattern for TARP in vivo mechano-activation upon delivery to injured discs.

To test the regenerative potential of TARP-mediated repair and Anakinra delivery, TARPs were tested in the goat cervical spine. Our in vivo study was approved by the University of Pennsylvania Institutional Animal Care and Use Committee (IACUC) and all surgeries followed the guidelines recommended by the committee. A total of 8 animals underwent surgery, consisting of annular injury of both C2-3 and C3-4 cervical discs followed by TARP repair of one level. Of the animals, n=4 received empty TARP repair (E-scaffold) and n=4 received Anakinra-loaded TARP repair (A-scaffold). Adjacent C4-5 levels were used as uninjured controls. Animals were sacrificed after 4 weeks, after which all motion segments underwent MRI T2 mapping, biomechanical testing, micro computed tomography imaging, and histological analysis. Due to level-to-level variations in MRI T2 relaxation times, level-matched uninjured controls from n=6 age-matched goats were used for MRI T2 map comparisons. One animal that received the A-scaffold was excluded from the study due to anatomical abnormalities found throughout the cervical spine. For each data set, outliers were identified as being outside 1.5× the interquartile range in Tukey box plots and were removed from the set before group comparisons.

### Nanofibrous Scaffold Fabrication

Dual component nanofibrous scaffolds composed of poly(e-caprolactone (PCL) (Shenzhn Esun Industrial Co., PCL 800C) and 200 kDa poly(ethylene oxide) (PEO) (Polysciences, 17503) were fabricated via electrospinning as previously described. Briefly, a 14.3% w/v solution of PCL dissolved in a 1:1 mixture of tetrahydrofuran and N,N-dimethylformamide was made. Separately, PEO was dissolved in 90% EtOH to yield a 10% w/v solution. 50:50 PCL/PEO sheets (300-350 μm thick) were fabricated by simultaneously collecting PCL and PEO onto a grounded, rotating mandrel to form aligned nanofibers (FIG. 36A).

PCL-PEO scaffold strips were cut to required dimensions, with the longest length along the direction of fiber alignment. Scaffolds were hydrated through a gradient of EtOH (100%, 70%, 50%, 30%, and 2× phosphate buffered solution (PBS). For experiments requiring sterility, sterile washes were performed under a tissue culture hood. Note that hydration of scaffolds removed the PEO fraction.

### Fabrication of Mechanically-Activated Microcapsules

MAMCs were fabricated through the generation of water-in-oil-in-water double emulsions utilizing three liquid phases flowed through a glass capillary microfluidic device as previously established (FIG. 29A). The inner phase was composed of a pH 7.4 aqueous solution containing 1 mg/mL bovine serum albumin (BSA) as a model drug or 2 mg/mL of purified interleukin-1 receptor antagonist (IL-1ra) known as Anakinra (Sobi, Kineret™), with 0.01% w/v AlexaFluor488-conjugated BSA (Invitrogen, A13100) for fluorescent visualization. Anakinra was purified from its base suspension solution using a 10 kDa filter (Millipore, UFC5010124), after which it was resuspended in PBS (pH7.4) containing AlexaFluor488-conjugated BSA to the desired concentration. Solution concentration was determined using the Pierce™ BCA Protein Assay (ThermoFisher, 23250). The middle phase was composed of 85:15 lactide: glycolide poly(D,L-lactide-co-glycolide) (PLGA) copolymer (Lactel, B6006-1), dissolved in chloroform and fluorescently labeled with 100 mg/mL of Nile Red (Sigma, N3013). The outer phase was composed of 2% w/v poly(vinyl alcohol) in water. Double emulsions were collected in a pH 12 0.1% BSA in PBS solution with 0.95M of NaCl and left to harden over 72 hrs., during which time the chloroform evaporated from the middle phase, leaving behind a solidified PLGA wall. After that time, MAMCs were collected, washed with PBS, and stored at 4° C.

Fabrication efficiency and MAMC dimensions were assessed via confocal microscopy (Nikon A1R+), at 4× and 60× magnification, respectively. % Full was calculated by dividing full MAMCs over total MAMCs (n=5 counts/MAMC batch of >1000 MAMCs/count). MAMC dimensions were calculated using ImageJ, with n=20 images/batch measured.

### Tension-Activated Annular Repair Patch Fabrication

TARPs were fabricated by melt-stamping MAMCs in between two hydrated PCL-PEO scaffold strips cut to dimensions required for each experiment (FIG. 29B). For assessment of stamping effect on MAMC patency, mechanical testing, local optical strain tracking, and fiber alignment/cellular morphology assessment, scaffold strips measuring 30 mm in length and 10 mm in width were used. For dynamic tensile loading, scaffold strips were cut to 60 mm in length and 10 mm in width. Finally, for in vivo studies, scaffold strips measuring 10 mm in length and 3.5 mm in width were used (FIG. 29C).

To melt-stamp scaffolds, 3D-printed metal stamps displaying a rhombus pattern with rhombus geometries that varied in longest rhombus diagonal length (δ) were used. Stamps with pattern δ=3, 4, and 5 mm were utilized (FIG. 36B). A large stamp (l: 20 mm, w: 10 mm) was used to create all TARPs except for those used in the in vivo study (FIG. 36B), which were assembled using a smaller stamp (l: 10 mm, w: 3.5 mm) displaying the 8=4 mm pattern. Stamps were heated at 80° C. on a heat plate. Hydrated scaffold strips were laid flat on a flat surface and excess PBS was aspirated carefully. MAMCs, resuspended at the concentration of interest in a volume of 1.5 mL PBS/mm² of scaffold (previously determined to not cause overflow during melt-stamping), were carefully pipetted over the flat scaffold strip. For all in vitro studies, TARPs were fabricated using a MAMC concentration of 700 MAMCs/mL. For in vivo implantation, empty TARPs (E-scaffold) were fabricated using 700 BSA-loaded MAMCs/mL while Anakinra-loaded TARPs (A-scaffold) were fabricated using 1,400 Anakinra-loaded MAMCs/mL to deliver 1 mg of Anakinra/TARP. After pipetting MAMCs onto the first scaffold strip, a second scaffold layer was carefully placed over the first, after removal of excess PBS, to cover MAMCs and the heated stamps were pressed over the scaffold-MAMCs-scaffold assembly for 3 seconds. Melt-stamped scaffolds were carefully detached from the stamp using tweezers and stored in PBS at 4° C.

### Assessment of Stamping Effect on MAMC Patency

To assess the effects of stamping on MAMC patency, TARPs scaffold layers were carefully peeled apart to enable visualization of encapsulated MAMCs. MAMC inner solution and outer shell were fluorescently imaged using a ZEISS Axio Zoom V16 and n=20 regions of interest/scaffold were visualized for n=4 TARPs/stamp pattern. % Full was quantified as described above.

### Mechanical Testing and Local Optical Strain Tracking

Before mechanical testing, the cross-sectional area of TARPs was measured using a custom-built laser device. Unstamped PEO-PCL scaffolds of comparable thickness were used as controls (thickness: ˜ 650 μm). TARPs were marked with black paint at the vertices of the rhombus patterns to enable local optical strain tracking of each rhombus during tensioning (FIG. 30A). Similarly, the unstamped control scaffolds were marked using a rhombus pattern. Small sections of sandpaper were superglued to both sides of each scaffold end (5 mm each end) to prevent slipping during testing. Scaffolds (n=5/type) were loaded in a universal test frame (Instron 5542, Instron, Norwood, MA) using custom grips at both ends and a 100N load cell. Loads were applied along the primary/longitudinal axis of the fibers. Specifically, a 60 sec. preload of 0.5N was applied, followed by 10 cycles of pre-conditioning at 0.5% strain at 0.1 Hz and ramp to failure at 0.1% strain/second. During loading, images of the marked scaffolds were captured with a high-definition camera at 1 frame/sec (FIG. 30A).

A custom post-processing script (Matlab 2021a, Mathworks, Natick, MA) was developed to calculate mechanical properties based on “grip-to-grip” displacements and optical strain measures. Optical strain measurements of the rhombus-shaped patterns were achieved by identifying the 4 nodes of a given rhombus and tracking the change in position of the centroid of each node on every image. Tracked nodes were used to calculate the local axial (ε_{y}) and transverse (εₓ) strains over the course of the mechanical test. Slopes of the linear regions of force-displacement were used to derive values for stiffness.

### Dynamic Mechanical Loading of TARPs

Using a custom-built bioreactor, n=4 TARPs/stamping pattern were dynamically tensile loaded at a time in a PBS bath to 6% strain at 1 Hz, for 300, 1,800, or 3,600 cycles. The effects of loading cycles on MAMC patency were visualized using an AxioZoom and quantified as described above. Areas in which the stamps melted the scaffolds and MAMCs upon contact were not included in the quantification.

Fiber Alignment and Cell Morphology Changes with Stamp Geometries

### Fiber Alignment and Dispersity Measurements

To assess fiber alignment changes with melt stamping, fiber autofluorescence was captured for unstamped PCL-PEO scaffolds and TARPs made with different stamp geometries (n=4 scaffolds/type, with n=4 ROIs/scaffold) under the DAPI channel using fluorescent confocal microscopy (Nikon A1R+, 20× magnification). Fiber alignment offset from 90° and alignment dispersity was calculated using the Directionality plugin in ImageJ. Fiber alignment maps overlayed over analyzed images were generated with the same plugin.

### Cell Alignment and Aspect Ratio Analysis

The effects of melt stamping and stamp geometries on AF cell morphology and organization were assessed through visualization and quantification of cellular alignment and aspect ratio. Human AF cells were cultured on fibronectin-coated unstamped scaffolds and TARPs with different stamp geometries (n=4/type) at cells/mm². After 6 days of culture, cells were washed 3× with PBS, after which they were fixed with 4% paraformaldehyde for 20 mins. at room temperature (RT). After fixation, the cells were washed 3× with PBS. Blocking was performed by adding blocking solution composed of 3% BSA in PBS for 1 hr. at RT. After blocking, cells were washed with PBS 3×, and cells were then stained with Draq5 (ThermoFisher, 62251) nuclear stain (1:1000) and Alexa Fluor 488 Phalloidin (ThermoFisher, A12379) (1:500) for 1 hr. at RT. After staining, cells were rinsed with PBS 2× and fluorescent confocal imaging was performed (Nikon A1R+, 20× magnification).

Cell aspect ratio and alignment angle were measured using CellProfiler, ver 3.1.9. Nuclei were viewed in n=30 ROIs/scaffold using the Draq5 signal and were segmented based on a minimum cross entropy algorithm with nuclei excluded below 5 and above 20 pixels. Nuclei were also excluded that were touching the image boundary. Cells were identified via propagation from the identified nuclei and segmented using a minimum cross entropy algorithm. Major and minor axes of the cells were measured, and their ratio (long to short axis) was presented as cell aspect ratio. The angle of the long axis of the cell was recorded to describe cell orientation compared to the scaffold fiber orientation.

### Bioactivity Assessment of MAMC-Encapsulated Anakinra

### In Vitro Culture and Treatment of Cells

To determine if the MAMC fabrication process affected the bioactivity of Anakinra after encapsulation, the effects of MAMC-encapsulated Anakinra were compared to those elicited by soluble Anakinra (FIG. 38). For this, human AF cells (Articular Engineering) (passage 2) were seeded at 6,000 cells/cm² in 12-well plates with basal media composed of DMEM supplemented with 10% fetal bovine serum and 1% Penicillin-Streptomycin-Fungizone. After 4 days (˜70% confluence), the cell media was removed, and cells received basal media with 10 ng/ml of interleukin 1β (IL-1β) (R&D Systems, 201/LB/CF). Media was additionally supplemented with soluble Anakinra or the contents of Anakinra-encapsulating MAMCs at 0, 10, 100, 500 or 1,000 ng/ml, with n=4 wells/group receiving each treatment. MAMC contents were extracted by crushing MAMCs using a pestle. Anakinra concentrations were measured from extracted contents using the Pierce™ BCA Protein Assay (ThermoFisher, 23250). Negative controls that did not receive any kind of IL-1β or Anakinra treatment were included. Media was replenished every 3 days, including all supplementing factors for each treatment group. After 6 days of treatment, cells were trypsinized, pelleted, and frozen.

### Gene Expression Analysis Via Quantitative Real Time RT-PCR

RNA isolation of digested samples was performed using the Directzol RNA Miniprep kit with DNAse-I treatment to remove trace DNA before RNA elution (Zymo Research, R2050). RNA was quantified via Nanodrop spectrophotometry. cDNA was synthesized using the SuperScript™ IV VILO Master Mix (Invitrogen, 11756050) according to the manufacturer's protocol. Relative quantitative RT-PCR was run using Fast SYBR™ Green Master Mix (ThermoFisher, 4385618) for 40 cycles with validated primers (primes listed elsewhere herein). Changes in gene expression were reported as 2^{-DDCT}. ΔC_{T} for a given sample and gene of interest was calculated by subtracting the C_{T} value for the housekeeping gene (GAPDH) from the C_{T} value for the gene of interest. For a given gene, ΔΔC_{T} for a sample was calculated by subtracting average ΔC_{T} for the untreated negative control samples from the ΔC_{T} calculated for the sample.

### Effects of Anakinra on Cell Viability

To test the effects of different Anakinra concentrations on cellular viability, human AF cells were cultured for 3 days in basal media, after which 0, 10, 100, 500 or 1,000 ng/mL of soluble Anakinra was added directly into the media, with n=5 wells/treatment. Media was replenished, including all supplementing factors for each treatment group, every 3 days. After 6 days, the media was removed and fresh media with alamarBlue™ Cell Viability Reagent (ThermoFisher, DAL1025) was added, following manufacturer's instructions. Empty wells also received the mixture as a background reference. After 4 hrs. of incubation at 37° C., fluorescence was measured using a spectrophotometer. Fluorescent intensity was calculated by subtracting the empty well reference measurement from each well measurement.

### In Vivo Assessment of TARPs Using a Goat Cervical Disc Annular Injury Model

### Injury Model & TARPs Implantation

Male (n=1) and female (n=7) skeletally mature (˜3 years of age), large frame goats were utilized. Under general anesthesia and using standard aseptic techniques, the animals underwent a surgical procedure at the C2-3 and C3-4 levels of the cervical spine to create an annular injury, with or without TARP implantation for repair of the induced injury (FIG. 31A). The ventral cervical spine was exposed using the anatomical plane between the trachea/esophagus and the carotid sheath. Intraoperative fluoroscopy was used to identify the vertebral bodies of C2 through C4. Soft tissues were bluntly and sharply dissected to expose the IVD of C2-3 and C3-4. Annular injury was induced, consisting of a cruciate partial thickness AF laceration (l: 5 mm, w: 2.5 mm, d: 4 mm), followed by full thickness puncture of the anterior AF through the middle of the X laceration (7 mm depth) with a 14G needle (outer diameter: 2.1 mm) (FIG. 31B-C). A TARP (δ=4 mm) loaded with BSA-encapsulating MAMCs (E-scaffold) or Anakinra-encapsulating MAMCs (A-scaffold) (l: 10 mm, w: 3.5 mm) was placed over the defect and sutured using 6-0 Gore-Tex monofilament suture (FIG. 31D). Sutures were placed in both corners of the cruciate annulotomy and the TARP was secured to each corner (FIG. 31E). Running sutures along the superior and inferior margins were used to further secure the TARP in place over the induced injury. The incision was then closed in layers and the animals recovered from anesthesia, after which animals were returned to standard housing consisting of 12 ft×12 ft stalls. Animals were euthanized at 4 weeks post-implantation, and the cervical spines harvested en bloc and stored frozen at −20° C. until thawing for MRI scans and mechanical testing analyses.

### Magnetic Resonance Imaging & Analysis

MRI scans of cervical spines were performed using a 3T scanner (Siemens Magnetom TrioTim). T2-weighted mid-sagittal images (5 mm slice thickness, 0.5 mm in plane resolution, TR/TE=4,540/123 ms) were obtained. A series of images for T2 mapping (6 echoes, TE=13 ms, 5 mm slice thickness, 0.5 mm in plane resolution) were also obtained.

Average T2 maps for each experimental group were generated using a custom MATLAB code. Due to level-to-level variations in AF and NP T2, C3-4 uninjured healthy control levels from goat cervical spines used in a separate study (n=6) were used for T2 comparisons. T2 measured along the central Y axis (y=0) of normalized T2 maps generated through the MATLAB code were plotted for each specimen to show changes in T2 signal along the central axis of each disc. Average AF and NP T2 values were obtained for each specimen by measuring rectangular and circular regions of interest for the AF and NP regions, respectively, on T2 maps using ImageJ.

### Motion Segment Biomechanical Testing & Analysis

Motion segments (vertebra-disc-vertebra units) were prepared for compression testing by carefully dissecting musculature around the disc and removing posterior and lateral boney elements with a hand saw. Ink spots were placed on the vertebral bone immediately distal and proximal to the disc for optical tracking during testing (FIG. 44A). Motion segments were then potted in a low melting temperature indium casting alloy (McMaster-Carr) in custom fixtures and subjected to a compression and creep testing protocol (Instron 5948) in a PBS bath with protease inhibitors. The testing protocol consisted of 20 cycles of compression from 0 to −100 N (0 to 0.14 MPa) followed by creep testing consisting of a 1 second step load to −0.14 MPa and a 60-minute hold.

A bi-linear fit of the 20th compression curve was performed in MATLAB to quantify toe and linear region modulus, as well as maximum compressive strain for each sample. Creep displacement was calculated by fitting the creep test to a 5-parameter viscoelastic constitutive model in MATLAB.

### MicroComputed Tomography Imaging & Analysis

To prepare motion segments for MicroComputed Tomography (μCT), samples were fixed for 7 days in formalin at 4° C. After fixation, samples were rinsed in PBS, wrapped in PBS-soaked gauze, and placed within the device for scanning. Motion segments were imaged at an isotropic 10 mm resolution using a Scanco Medical μCT50.

Cranial and caudal bony endplates (defined as the region between the intervertebral disc and growth plate) were manually contoured (FIG. 39A) and bone morphometry parameters, including bone volume over total volume (BV/TV), trabecular number (Tb. N.), and trabecular spacing (Tb. Sp) were calculated using the Scanco Medical Analyzer software. Regions where osteolysis was apparent on the cranial and caudal bony endplates were manually contoured and osteolysis volume was calculated. Scanco Medical Visualizer software was used to generate 3D reconstructions of specimen scans with color-coded display of bone density.

### Histological Assessment of Disc Composition and Structure

Motion segments were decalcified (Formical-2000, Decal Chemical Corporation, Tallman, NY) and processed through paraffin. 10 μm sections around the mid-sagittal plane were collected. For all stains performed, sections from all experimental groups were stained simultaneously. Sections were co-stained for glycosaminoglycans and collagens using Alcian Blue and Picrosirius Red (AB/PSR), respectively. NP Alcian Blue staining intensity was measured using a circular region of interest in ImageJ. For the visualization of microscopic anatomy and cellular morphology, sections were stained with hematoxylin and eosin (H&E). To visualize collagen fibrils, elastin, bone, other hyaline structures, and cells, a one-step Mallory-Heidenhain (MH) stain was used (FIG. 34). Collagenous scar infiltration area along the anterior and posterior AF was measured in the red channel using thresholds applied with ImageJ.

Statistical analyses were performed using Prism 9 (Graph Pad Software Inc.), with significance defined as p-value<0.05. Quantitative data is presented as mean±standard deviation (s.d.) or mean±standard error of the mean (SEM). The Shapiro-Wilk normality test was used to determine the need for non-parametric testing (alpha=0.05). For each data set, outliers were identified as being outside 1.5× the interquartile range in Tukey box plots and were removed from the set before group comparisons. For comparisons between stamping patterns (MAMC patency post-stamping, bulk scaffold biomechanical properties, local strains, fiber alignment and dispersity, and cellular alignment and aspect ratio analyses), one-way ANOVA with Tukey's multiple comparisons post-hoc analysis was employed. Similarly, for Alamar Blue and qPCR analyses for different concentrations of Anakinra, a one-way ANOVA with Tukey's multiple comparisons post-hoc analysis was used. MAMC mechano-activation with dynamic tensile loading was analyzed using a two-way ANOVA with Tukey's multiple comparisons post-hoc test. To characterize the effect of the induced annular injury, uninjured controls and injury groups were compared using an unpaired t-test with Welch's correction. AF and NP T2 signal, disc biomechanical properties, bone morphological characteristics, anterior and posterior disc scar infiltration/remodeling area, and NP Alcian Blue intensity were analyzed using one-way ANOVA with Tukey's multiple comparisons post-hoc analysis. Osteolysis measurements were compared using a non-parametric one-way ANOVA with Dunn's multiple comparisons.

### Primer Sequences for Quantitative RT-PCR.

| Target | Forward Primer | Reverse Primer |
|---|---|---|
| GAPDH | TGGCCTTCCGTGTCCCCACT | GGCCCTCCGACGCCTGCTTC |
| | GCCA | ACCA |
| MMP3 | GCTGTTTTTGAAGAATTTGG | GCACAGGCAGGAGAAAACGA |
| | GTTC | |
| LCN2 | TCACCTCCGTCCTGTTTAGG | CGAAGTCAGCTCCTTGGTTC |

### Aspects

The following Aspects are illustrative only and do not limit the scope of the present disclosure or the appended claims. Any part or parts of any Aspect can be combined with any part or parts of any other Aspect or Aspects.

Aspect 1. A localized therapeutic delivery article, comprising: a first fibrous layer comprising a first plurality of nanofibers; and a second fibrous layer comprising a second plurality of nanofibers, the first fibrous layer and the second fibrous layer being sealed to one another so as to define at least one sealed compartment therebetween, the article optionally comprising a first population of mechanically-responsive delivery particles configured to rupture upon exposure to a first rupture force, the first population of mechanically responsive delivery particles being disposed within at least one sealed compartment. A sealed compartment can have a cross-sectional shape that is, e.g., oval, rhombic, or otherwise shaped. Cross-sectional shapes that have aspect ratios of other than 1 (e.g., non-circular cross-sectional shapes) are considered particularly suitable. The fibrous layers can be sufficiently pervious such that the contents of a ruptured delivery particle can pass through the layers. As an example, following application of sufficient force to rupture a mechanically-responsive delivery particle having a therapeutic contained therein, the liberated therapeutic can then exit the compartment.

The article can be, e.g., in the form of a patch or other portion. The article can be, e.g., circular, polygonal, or otherwise shaped.

Aspect 2. The article of Aspect 1, wherein the nanofibers of the first fibrous layer are aligned along a first direction, wherein the nanofibers of the second fibrous layer are aligned along a second direction, and wherein the first direction and the second direction are parallel to one another.

Aspect 3. The article of Aspect 1, wherein the nanofibers of the first fibrous layer are aligned along a first direction, wherein the nanofibers of the second fibrous layer are aligned along a second direction, and wherein the first direction and the second direction are at an angle to another. The angle can be, e.g., from 1 to 179.9 degrees, from 1 to 150 degrees, from 1 to 120 degrees, from 1 to 100 degrees, from 1 to 90 degrees, from 1 to 75 degrees, from 1 to 60 degrees, from 1 to 45 degrees, from 1 to 30 degrees, from 1 to 20 degrees, from 1 to 15 degrees, or from 1 to 10 degrees.

Aspect 4. The article of any one of Aspects 1-3, wherein at least one of the first plurality of nanofibers and the second plurality of nanofibers comprises a biocompatible polymer. As explained elsewhere herein, at least one of the first and second plurality of nanofibers can be aligned nanofibers.

Aspect 5. The article of Aspect 4, wherein the biocompatible polymer comprises polycaprolactone (PCL), polyethylene oxide (PEO), poly(ester urethane), poly(ester urethane) urea, poly(L-lactic acid), poly(D, L-lactic acid), poly(lactic-co-glycolic acid), gelatin, collagen, chitosan, hyaluronic acid, silk, polyethylene glycol, polydiaxanone-elastin, poly(ester-urethane) urea-collagen, poly(p-diaxanone-co-L-lactide)-block-poly(ethylene glycol), poly(L-lactide-co-ε-caprolactone), collagen-poly(ethylene oxide), or any combination thereof. As one non-limiting example, PCL-PEO polymer fibers are considered suitable.

Aspect 6. The article of any one of Aspects 1-5, wherein a sealed compartment defines a polygonal cross-section, the polygonal cross-section optionally having an aspect ratio other than 1.

Aspect 7. The article of any one of Aspects 1-6, comprising a plurality of sealed compartments, the plurality of sealed compartments optionally being present in a periodic arrangement. The compartments can all be of the same size and/or the same aspect ratio, but this is not a requirement, as an article can comprise compartments of different sizes and/or aspect ratios.s

Aspect 8. The article of any one of Aspects 1-7, wherein a sealed compartment defines a cross-sectional dimension in the range of from about 0.5 to about 10 mm, optionally in the range of from about 1 to about 9 mm, or from about 2 to about 8 mm, or from about 3 to about 7 mm, or from about 4 to about 6 mm.

Aspect 9. The article of Aspect 1, further comprising a first population of mechanically-responsive delivery particles configured to rupture upon exposure to a first rupture force, the first population of mechanically responsive delivery particles being disposed within at least one sealed compartment.

Aspect 10. The article of Aspect 9, wherein the first population of mechanically-responsive delivery particles comprises at least one therapeutic therein.

Aspect 11. The article of Aspect 10, wherein the at least one therapeutic comprises an antibody, a cytokine, a receptor antagonist, an analgesic, a growth factor, a small molecule inhibitor, a protein inhibitor, an enzyme, or any combination thereof. As some non-limiting examples, a growth factor can be, e.g., transforming growth factor beta 3, platelet derived growth factor, and the like. Example inhibitors include, e.g., caspase inhibitors (small molecules) to prevent apoptosis, tissue inhibitors of metalloproteinases (TIMPs-proteins), and the like.

Aspect 12. The article of Aspect 10, wherein the therapeutic comprises Tanezumab, Anakinra, or both.

Aspect 13. The article of any one of Aspects 9-12, wherein the article is configured to effect rupture of at least some of the first population of delivery particles upon the article experiencing a strain of about 1% to about 35% with from 1 to 1,000,000 loading cycles.

For example, an article can be configured to effect rupture of at least some of the first population of delivery particles upon the article experiencing a strain of from about 1% to about 35%, or a strain of from about 2% to 30%, or a strain of from about 3% to about 25%, or a strain or from about 4% to about 20%, or from about 5% to about 15%, or from about 6% to about 12%, over from 1 to 1,000 loading cycles, or from 10 to 750 loading cycles, or from 25 to 500 loading cycles, or from 50 to 250 loading cycles.

As a non-limiting example, the article can be configured to effect rupture of at least some of the first population of delivery particles upon the article experiencing a strain of about 6 percent with 3,600 loading cycles. The strain can be in a plane parallel to a plane of the first or second fibrous layers; the strain can also be in a plane parallel to a plane in which a sealed compartment lies.

Aspect 14. The article of any one of Aspects 9-13, further comprising a second population of mechanically-responsive delivery particles configured to rupture upon exposure to a second rupture force, the second population of mechanically responsive delivery particles being disposed within at least one sealed compartment.

Aspect 15. The article of Aspect 14, wherein the second rupture force differs from the first rupture force. Thus, an article can include two populations of mechanically-responsive delivery particles, one of which populations ruptures more easily than the other of the populations. As an example, a first population can be configured to rupture following about 500 loading cycles, and the second population can be configured to rupture following about 1500 loading cycles.

Aspect 16. The article of any one of Aspects 13-15, wherein the second population of mechanically-responsive delivery particles differs from the first population of mechanically-responsive delivery particles in terms of one or more of composition, size, and contents. Thus, an article can include, for example, a first population of mechanically-responsive delivery particles that releases their contents between 1 and 5 days after introduction to a subject, and a second population of mechanically-responsive delivery particles that releases their contents between 6 and 10 days after introduction to the subject. In this way, an article can be configured to effect release of one or more therapeutics over extended periods of time, and can even be configured to effect release of different therapeutics at the same time or at different times.

Aspect 17. A method, comprising introducing an article according to any one of Aspects 1-16 to a subject, the introducing optionally being to an annulus fibrosus of the subject. The introducing can be by suture, for example. An article can also be introduced so as to effect a tension on the tissue to which the article is attached. As one non-limiting example, an article can be sutured to an annulus fibrosus so as to give rise to a tension in the annulus fibrosus.

Aspect 18. The method of Aspect 17, wherein the subject is mammalian.

Aspect 19. The method of Aspect 18, wherein the subject is human.

Aspect 20. A method, comprising causing the introduction of an article according to any one of Aspects 1-16 to a subject, the introduction optionally being to the annulus fibrosus of the subject.

Aspect 21. A method, comprising causing application of a strain to an article according to any one of Aspects 1-16. The application of strain can be effected by, e.g., suturing the article in place.

Aspect 22. A method, comprising application of a strain to any article according to any one of Aspects 9-16 so as to effect rupture of at least some of the first population of delivery particles. The application of strain can be effected by, e.g., movement of a subject to whom the article has been introduced. Such movement can be physiological movement, e.g., daily movement, movement effected by physical therapy, and the like.

Aspect 23. A method, comprising fabricating an article according to any one of Aspects 1-16.

Aspect 24. A method, comprising: with a first fibrous layer comprising a first plurality of nanofibers and a second fibrous layer comprising a second plurality of nanofibers, sealing the first fibrous layer and the second fibrous layer to one another so as to define at least one sealed compartment therebetween.

Aspect 25. The method of Aspect 24, further comprising effecting placement of a first population of mechanically-responsive delivery particles in one or more of the at least one sealed compartments

Aspect 26. The method of any one of Aspects 24-25, wherein the sealing is effected by application of a stamp.

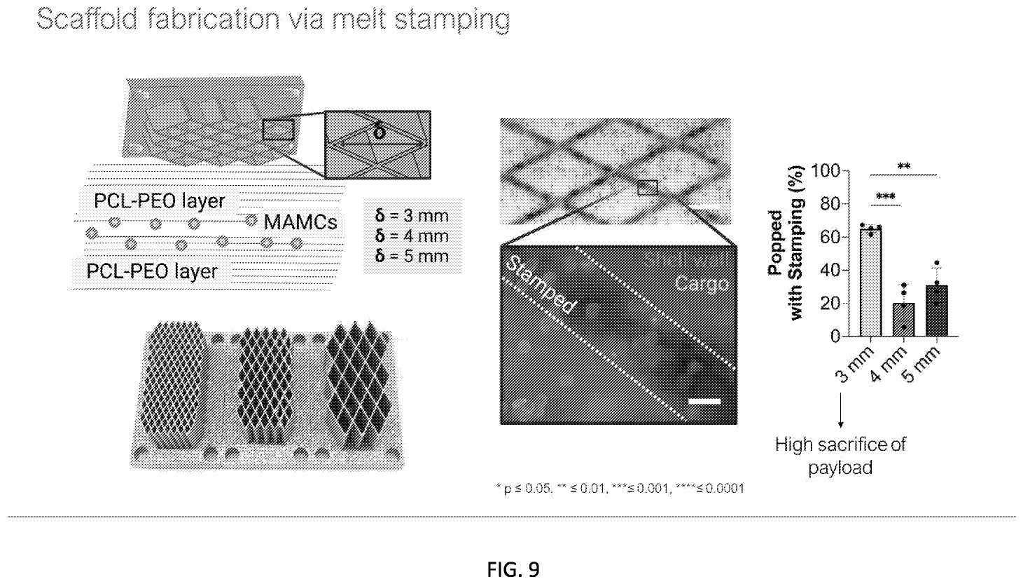

Aspect 27. The method of Aspect 26, wherein application of the stamp defines a profile of at least one sealed compartment. As an example, the stamp can bear a pattern that corresponds to the profile of one or more sealed compartments; as shown in, e.g., FIGS. 1, 5, 9, 29, and 36 and their associated disclosure, a stamp can bear a pattern, e.g., a rhombic pattern, which rhombic in turn gives rise to a corresponding pattern of sealed compartments in the finished article.

Aspect 28. The method of any one of Aspects 24-27, wherein the nanofibers of the first fibrous layer are aligned along a first direction and wherein the nanofibers of the second fibrous layer are aligned along a second direction.

Aspect 29. The method of Aspect 28, wherein the first direction and the second direction are parallel to one another.

Aspect 30. The method of Aspect 28, wherein the first direction and the second direction are at an angle to another.

Aspect 31. The method of any one of Aspects 24-30, further comprising electrospinning at least one of the first plurality of nanofibers and the second plurality of nanofibers.

### REFERENCES

Pang, X., Jiang, Y., Xiao, Q., Leung, A. W., Hua, H., and Xu, C. (2016) PH-responsive polymer-drug conjugates: Design and progress. J. Control. Release, 222, 116-129.
Wang, Y., and Kohane, D. S. (2017) External triggering and triggered targeting strategies for drug delivery. Nat. Rev. Mater., 2.
Jo, Y. K., and Lee, D. (2019) Biopolymer Microparticles Prepared by Microfluidics for Biomedical Applications. Small, 16 (9), 1903736.
Burt, H. M., Tsallas, A., Gilchrist, S., and Liang, L. S. (2009) Intra-articular drug delivery systems: Overcoming the shortcomings of joint disease therapy. Expert Opin. Drug Deliv., 6 (1), 17-26.
Rai, M. F., and Pham, C. T. (2018) Intra-articular drug delivery systems for joint diseases. Curr. Opin. Pharmacol., 40, 67-73.
Vega, S. L., Kwon, M. Y., and Burdick, J. A. (2017) Recent advances in hydrogels for cartilage tissue engineering. Eur. Cells Mater., 33, 59-75.
Geiger, B. C., Wang, S., Padera, R. F., Grodzinsky, A. J., and Hammond, P. T. (2018) Cartilage-penetrating nanocarriers improve delivery and efficacy of growth factor treatment of osteoarthritis. Sci. Transl. Med., 10 (469).
Mohanraj, B., Duan, G., Peredo, A., Kim, M., Tu, F., Lee, D., Dodge, G. R., and Mauck, R. L. (2019) Mechanically Activated Microcapsules for “On-Demand” Drug Delivery in Dynamically Loaded Musculoskeletal Tissues. Adv. Funct. Mater., 29 (15), 1807909.
Peredo, A. P., Jo, Y. K., Duan, G., Dodge, G. R., Lee, D., and Mauck, R. L. (2021) Mechano-activated biomolecule release in regenerating load-bearing tissue microenvironments. Biomaterials, 265.
Kokubo, Y et al., J Neurosurg, 9 (3): 285-95, 2008.
Peredo, A P et al., Tissue Eng Part B Rev, 27 (4): 295-312, 2021.
Peredo, A P et al., JOR Spine, 4 (1): e1133, 2021.
Johnson, Z I et al., Eur Cell Mater, 30:104-16, 2015.
Abe Y et al., ORS Annual Conference 2006, #1194
Ashinsky, B G et al., Acta Biomater, 111:232-241, 2020.
Baker, B M et al., Biomaterials, 29 (15): 2348-58, 2008.
l. M. D. Humzah, R. W. Soames, Human intervertebral disc: Structure and function Anat. Rec. 220, 337-356 (1988).
K. Lundon, K. Bolton, Structure and Function of the Lumbar Intervertebral Disk in Health, Aging, and Pathologic Conditions. J. Orthop. Sport. Phys. Ther. 31, 291-306 (2001).
S. C. Humphreys, J. C. Eck, C. Tennessee, Clinical evaluation and treatment options for herniated lumbar disc. Am. Fam. Physician 59, 575-582 (1999).
P. Goupille, M. I. V. Jayson, J. P. Valat, A. J. Freemont, The role of inflammation in disk herniation-associated radiculopathy. Semin. Arthritis Rheum. 28, 60-71 (1998).
G. D. Schroeder, C. A. Guyre, A. R. Vaccaro, The epidemiology and pathophysiology of lumbar disc herniations. Semin. Spine Surg. 28, 2-7 (2016).
A. Aichmair, J. Du, J. Shue, G. Evangelisti, A. Sama, A. Hughes, D. Lebl, J. Burket, F. Cammisa, F. Girardi, Microdiscectomy for the Treatment of Lumbar Disc Herniation: An Evaluation of Reoperations and Long-Term Outcomes. Evid. Based. Spine. Care. J. 05, 077-086 (2014).
J. M. Vinas-Rios, M. Sanchez-Aguilar, F. A. Medina Govea, V. Von Beeg-Moreno, F. Meyer, Incidence of early postoperative complications requiring surgical revision for recurrent lumbar disc herniation after spinal surgery: A retrospective observational study of 9,310 patients from the German Spine Register. Patient Saf. Surg. 12, 1-4 (2018).
M. Gronblad, J. Virri, J. Tolonen, S. Seitsalo, E. Kaapa, J. Kankare, P. Myllynen, E. O. Karaharju, A controlled immunohistochemical study of inflammatory cells in disc herniation tissue. Spine (Phila. Pa. 1976). 19, 2744-2751 (1994).
K. Jimbo, J. S. Park, K. Yokosuka, K. Sato, K. Nagata, Positive feedback loop of interleukin-1beta upregulating production of inflammatory mediators in human intervertebral disc cells in vitro. J. Neurosurg. Spine 2, 589-595 (2005).
H. Kim, J. Y. Hong, J. Lee, W. J. Jeon, I. H. Ha, IL-1β promotes disc degeneration and inflammation through direct injection of intervertebral disc in a rat lumbar disc herniation model. Spine J. 21, 1031-1041 (2021).
D. Purmessur, A. J. Freemont, J. A. Hoyland, Expression and regulation of neurotrophins in the nondegenerate and degenerate human intervertebral disc. Arthritis Res. Ther. 10 (2008), doi: 10.1186/ar2487.
H. E. Gruber, B. Jones, E. Marrero, E. N. Hanley, Proinflammatory Cytokines IL-1β and TNF-α Influence Human Annulus Cell Signaling Cues for Neurite Growth: In Vitro Coculture Studies. Spine (Phila. Pa. 1976). 42, 1529-1537 (2017).
H. E. Gruber, G. L. Hoelscher, S. Bethea, E. N. Hanley, Interleukin 1-beta upregulates brain-derived neurotrophic factor, neurotrophin 3 and neuropilin 2 gene expression and NGF production in annulus cells. Biotech. Histochem. 87, 506-511 (2012).
J.-N. Gouze, E. Gouze, G. D. Palmer, V. S. Liew, A. Pascher, O. B. Betz, T. S. Thornhill, C. H. Evans, A. J. Grodzinsky, S. C. Ghivizzani, A comparative study of the inhibitory effects of interleukin-1 receptor antagonist following administration as a recombinant protein or by gene transfer. Arthritis Res. Ther. 5, R301 (2003).
J. T. Martin, C. M. Collins, K. Ikuta, R. L. Mauck, D. M. Elliott, Y. Zhang, D. G. Anderson, A. R. Vaccaro, T. J. Albert, V. Arlet, H. E. Smith, Population average T2 MRI maps reveal quantitative regional transformations in the degenerating rabbit intervertebral disc that vary by lumbar level. J. Orthop. Res. 33, 140-148 (2015).
S. E. Gullbrand, B. G. Ashinsky, J. T. Martin, S. Pickup, L. J. Smith, R. L. Mauck, H. E. Smith, Correlations between quantitative T2 and T1ρ MRI, mechanical properties and biochemical composition in a rabbit lumbar intervertebral disc degeneration model. J. Orthop. Res. 34, 1382-1388 (2016).
B. Alkhatib, D. H. Rosenzweig, E. Krock, P. J. Roughley, L. Beckman, T. Steffen, M. H. Weber, J. A. Ouellet, L. Haglund, Acute mechanical injury of the human intervertebral disc: link to degeneration and pain. Eur. Cell. Mater. 28, 98-110; discussion 110-1 (2014).
C. Court, J. R. Chin, E. Liebenberg, O. K. Colliou, J. C. Lotz, Biological and mechanical consequences of transient intervertebral disc bending. Eur. Spine J. 16, 1899-1906 (2007).
E. D. Bonnevie, S. E. Gullbrand, B. G. Ashinsky, T. K. Tsinman, D. M. Elliott, P. hsiu G. Chao, H. E. Smith, R. L. Mauck, Aberrant mechanosensing in injured intervertebral discs as a result of boundary-constraint disruption and residual-strain loss. Nat. Biomed. Eng. 3, 998-1008 (2019).
J. Qin, X. He, D. Wang, P. Qi, L. Guo, S. Huang, X. Cai, H. Li, R. Wang, Artificial cervical vertebra and intervertebral complex replacement through the anterior approach in animal model: a biomechanical and in vivo evaluation of a successful goat model. PLOS One 7 (2012), doi: 10.1371/JOURNAL.PONE.0052910.
H. J. Wilke, L. Ressel, F. Heuer, N. Graf, S. Rath, Can prevention of a reherniation be investigated? establishment of a herniation model and experiments with an anular closure device. Spine (Phila. Pa. 1976). 38 (2013), doi: 10.1097/BRS.0b013e31828ca4bc.
S. L. Parker, G. Grahovac, D. Vukas, M. Vilendecic, D. Ledic, M. J. Mcgirt, E. J. Carragee, Effect of an annular closure device (Barricaid) on same-level recurrent disk herniation and disk height loss after primary lumbar discectomy. Clin. Spine Surg. 29, 454-460 (2016).
Food and Drug Administration, PMA P160050B: FDA Summary of Safety and Effectiveness Data (2019; https://www.accessdata.fda.gov/cdrh_docs/pdf16/P160050B.pdf).
A. P. Peredo, S. Gullbrand, H. E. Smith, R. L. Mauck, Putting the Pieces in Place: Mobilizing Cellular Players to Improve Annulus Fibrosus Repair. Tissue Eng. Part B Rev., ten. TEB.2020.0196 (2020).
R. G. Long, S. J. Ferguson, L. M. Benneker, D. Sakai, Z. Li, A. Pandit, D. W. Grijpma, D. Eglin, S. Zeiter, T. Schmid, U. Eberli, D. Nehrbass, T. Di Pauli von Treuheim, M. Alini, J. C. Iatridis, S. Grad, Morphological and biomechanical effects of annulus fibrosus injury and repair in an ovine cervical model. JOR SPINE (2019), doi: 10.1002/jsp2.1074.
S. R. Sloan, C. Wipplinger, S. Kirnaz, R. Navarro-Ramirez, F. Schmidt, D. McCloskey, T. Pannellini, A. Schiavinato, R. Härtl, L. J. Bonassar, Combined nucleus pulposus augmentation and annulus fibrosus repair prevents acute intervertebral disc degeneration after discectomy. Sci. Transl. Med. 12 (2020), doi: 10.1126/scitranslmed.aay2380.
S. R. Sloan, M. Lintz, I. Hussain, R. Hartl, L. J. Bonassar, Biologic Annulus Fibrosus Repair: A Review of Preclinical In Vivo Investigations. Tissue Eng. Part B Rev. 24, 179-190 (2018).
A. J. Michalek, M. G. Gardner-Morse, J. C. Iatridis, Large residual strains are present in the intervertebral disc annulus fibrosus in the unloaded state. J. Biomech. 45, 1227-1231 (2012).
N. Kos, L. Gradisnik, T. Velnar, A Brief Review of the Degenerative Intervertebral Disc Disease. Med. Arch. (Sarajevo, Bosnia Herzegovina) 73, 421-424 (2019).
L. A. Setton, J. Chen, Mechanobiology of the intervertebral disc and relevance to disc degeneration. J. Bone Joint Surg. Am. 88 Suppl 2, 52-57 (2006).
S. Kirnaz, C. Capadona, M. Lintz, K. Byumsu, R. Yerden, J. L. Goldberg, B. Medary, F. Sommer, L. B. McGrath, L. J. Bonassar, R. Hartl, Pathomechanism and Biomechanics of Degenerative Disc Disease: Features of Healthy and Degenerated Discs. Int. J. Spine Surg. 15, 10-25 (2021).
D. Hampton, G. Laros, R. McCarron, D. Franks, Healing potential of the anulus fibrosus. Spine (Phila. Pa. 1976). 14, 398-401 (1989).
J. A. Key, L. T. Ford, Experimental intervertebral-disc lesions. J. Bone Joint Surg. Am. 30, 621-630 (1948).
J. W. Smith, R. Walmsley, Experimental incision of the intervertebral disc. J. Bone Joint Surg. Br. 33 B, 612-625 (1951).
M. H. Lee, K. C. Hribar, T. Brugarolas, N. P. Kamat, J. A. Burdick, D. Lee, Drug Delivery: Harnessing Interfacial Phenomena to Program the Release Properties of Hollow Microcapsules (Adv. Funct. Mater. 1/2012). Adv. Funct. Mater. 22, 130-130 (2012).
M. Favata, Scarless healing in the fetus: Implications and strategies for postnatal tendon repair. Diss. available from ProQuest (2006) (available at https://repository.upenn.edu/dissertations/AAI3246156).
B. M. Baker, R. P. Shah, A. H. Huang, R. L. Mauck, Dynamic tensile loading improves the functional properties of mesenchymal stem cell-laden nanofiber-based fibrocartilage. Tissue Eng. Part A 17, 1445-55 (2011).
S. E. Gullbrand, B. G. Ashinsky, E. D. Bonnevie, D. H. Kim, J. B. Engiles, L. J. Smith, D. M. Elliott, T. P. Schaer, H. E. Smith, R. L. Mauck, Long term mechanical function and integration of an implanted tissue engineered intervertebral disc. Sci. Transl. Med. 10 (2018), doi: 10.1126/SCITRANSLMED.AAU0670.
J. T. Martin, D. J. Gorth, E. E. Beattie, B. D. Harfe, L. J. Smith, D. M. Elliott, Needle puncture injury causes acute and long-term mechanical deficiency in a mouse model of intervertebral disc degeneration. J. Orthop. Res. 31, 1276-1282 (2013).
J. E. Cason, A Rapid One-Step Mallory-Heidenhain Stain for Connective Tissue. Stain Technol. 25, 225-226 (1950).
Z. Y. Sun, Y. T. Liu, H. Liang, Y. Li, D. G. Wang, J. W. Tian, Interleukin-1β exacerbates the catabolic effects of human nucleus pulposus cells through activation of the Nuclear Factor kappa B signaling pathway under hypoxic conditions. Eur. Rev. Med. Pharmacol. Sci. 22, 7129-7139 (2018).
S. L. Kim, S. T. Lee, I. S. Min, Y. R. Park, J. H. Lee, D. G. Kim, S. W. Kim, Lipocalin 2 negatively regulates cell proliferation and epithelial to mesenchymal transition through changing metabolic gene expression in colorectal cancer. Cancer Sci. 108, 2176-2186 (2017).

1. A localized therapeutic delivery article, comprising:
a first fibrous layer comprising a first plurality of nanofibers; and
a second fibrous layer comprising a second plurality of nanofibers,
the first fibrous layer and the second fibrous layer being sealed to one another so as to define at least one sealed compartment therebetween,
the article optionally comprising a first population of mechanically-responsive delivery particles configured to rupture upon exposure to a first rupture force, the first population of mechanically responsive delivery particles being disposed within at least one sealed compartment.

2. The article of claim 1, wherein the nanofibers of the first fibrous layer are aligned along a first direction, wherein the nanofibers of the second fibrous layer are aligned along a second direction, and wherein the first direction and the second direction are parallel to one another.

3. The article of claim 2, wherein the nanofibers of the first fibrous layer are aligned along a first direction, wherein the aligned nanofibers of the second fibrous layer are aligned along a second direction, and wherein the first direction and the second direction are at an angle to another.

4. The article of claim 1, wherein at least one of the first plurality of nanofibers and the second plurality of nanofibers comprises a biocompatible polymer.

5. The article of claim 4, wherein the biocompatible polymer comprises polycaprolactone (PCL), polyethylene oxide (PEO), poly(ester urethane), poly(ester urethane) urea, poly(L-lactic acid), poly(D, L-lactic acid), poly(lactic-co-glycolic acid), gelatin, collagen, chitosan, hyaluronic acid, silk, polyethylene glycol, polydiaxanone-elastin, poly(ester-urethane) urea-collagen, poly(p-diaxanone-co-L-lactide)-block-poly(ethylene glycol), poly(L-lactide-co-ε-caprolactone), collagen-poly(ethylene oxide), or any combination thereof.

6. The article of claim 1, wherein a sealed compartment defines a polygonal cross-section, the polygonal cross-section optionally having an aspect ratio other than 1.

7. The article of claim 1, comprising a plurality of sealed compartments, the plurality of sealed compartments optionally being present in a periodic arrangement.

8. The article of claim 1, wherein a sealed compartment defines a cross-sectional dimension in the range of from about 0.5 to about 10 mm, optionally in the range of from about 2 to about 7 mm.

9. The article of claim 1, further comprising a first population of mechanically-responsive delivery particles configured to rupture upon exposure to a first rupture force, the first population of mechanically responsive delivery particles being disposed within at least one sealed compartment.

10. The article of claim 9, wherein the first population of mechanically-responsive delivery particles comprises at least one therapeutic therein.

11. The article of claim 10, wherein the at least one therapeutic comprises an antibody, a cytokine, a receptor antagonist, an analgesic, a growth factor, a small molecule inhibitor, a protein inhibitor), an enzyme, or any combination thereof.

12. The article of claim 10, wherein the therapeutic comprises Tanezumab, Anakinra, or both.

13. The article of claim 9, wherein the article is configured to effect rupture of at least some of the first population of delivery particles upon the article experiencing a strain of 1% to about 35% with from 1 to 1,000,000 loading cycles.

14. The article of claim 9, further comprising a second population of mechanically-responsive delivery particles configured to rupture upon exposure to a second rupture force, the second population of mechanically responsive delivery particles being disposed within at least one sealed compartment.

15. The article of claim 14, wherein the second rupture force differs from the first rupture force.

16. The article of claim 14, wherein the second population of mechanically-responsive delivery particles differs from the first population of mechanically-responsive delivery particles in terms of one or more of composition, size, and contents.

17. A method, comprising introducing an article according to claim 1 to a subject, the introducing optionally being to an annulosis fibrosis of the subject.

18. The method of claim 17, wherein the subject is mammalian.

19. The method of claim 18, wherein the subject is human.

20. A method, comprising causing the introduction of an article according to claim 1 to a subject, the introduction optionally being to the annulus an annulosis fibrosus of the subject.

21. A method, comprising causing application of a strain to an article according to claim 1.

22. A method, comprising application of a strain to any article according to claim 9 so as to effect rupture of at least some of the first population of delivery particles.

23. A method, comprising fabricating an article according to claim 1.

24. A method, comprising:
with a first fibrous layer comprising a first plurality of nanofibers and a second fibrous layer comprising a second plurality of nanofibers,
sealing the first fibrous layer and the second fibrous layer to one another so as to define at least one sealed compartment therebetween.

25. The method of claim 24, further comprising effecting placement of a first population of mechanically-responsive delivery particles in one or more of at least one sealed compartments.

26. The method of claim 24, wherein the sealing is effected by application of a stamp.

27. The method of claim 26, wherein application of the stamp defines a profile of at least one sealed compartment.

28. The method of claim 24, wherein the nanofibers of the first fibrous layer are aligned along a first direction and wherein the nanofibers of the second fibrous layer are aligned along a second direction.

29. The method of claim 28, wherein the first direction and the second direction are parallel to one another.

30. The method of claim 28, wherein the first direction and the second direction are at an angle to another.

31. The method of claim 24, further comprising electrospinning at least one of the first plurality of nanofibers and the second plurality of nanofibers.
